(19) United States
(12) Patent Application Publication
(10) Pub. No.: US 2026/0096561 A1
(43) Pub. Date: Apr. 9, 2026

(54) BACTERIOPHAGE HAVING BACTERIOLYTIC ACTIVITY AGAINST XANTHOMONAS BACTERIA

(71) Applicants: KANEKA CORPORATION, Osaka (JP); RAKUNO GAKUEN UNIVERSITY, Hokkaido (JP); MITSUI & CO., LTD., Tokyo (JP)

(72) Inventors: Shinichi Yoshida, Shizuoka (JP); Nobuhiko Dojun, Shizuoka (JP); Hidetomo Iwano, Hokkaido (JP); Hitoshi Kudo, Tokyo (JP)

(73) Assignees: KANEKA CORPORATION, Osaka (JP); RAKUNO GAKUEN UNIVERSITY, Hokkaido (JP); MITSUI & CO., LTD., Tokyo (JP)

(21) Appl. No.: 19/402,216

(22) Filed: Nov. 26, 2025

Related U.S. Application Data
(63) Continuation of application No. PCT/JP2024/019669, filed on May 29, 2024.

(30) Foreign Application Priority Data
May 31, 2023 (JP) 2023-089380

(51) Int. Cl. A01N 63/40; A01P 1/00; C12Q 1/04

(52) U.S. Cl. CPC A01N 63/40; A01P 1/00; C12Q 1/04

(57) ABSTRACT

Methods are provided for controlling a plant disease caused by a bacterium of genus Xanthomonas, comprising contacting a bacteriophage to a target plant, and for identifying a bacterium of the genus Xanthomonas, involving: culturing a test bacterium isolated from a plant tissue affected by a plant disease to obtain a culture; mixing the culture with a bacteriophage to obtain a mixture; culturing the mixture under a predetermined condition; and determining that the test bacteria are bacteria of the genus Xanthomonas when the test bacteria are lysed after the mixture-culturing step.

### SEQUENCE LISTING

This application contains a sequence listing in computer readable form (File name: PH-10246-PCT_SequenceListing.xml; date of creation: Oct. 16, 2025; File size: 874,999 bytes) which is incorporated herein by reference in its entirety and forms part of the disclosure.

### TECHNICAL FIELD

One or more embodiments of the present invention relates to a bacteriolytic agent comprising a bacteriophage, a plant disease control composition containing the bacteriolytic agent, and a method for controlling a plant disease.

### BACKGROUND

Bacteriophage (often abbreviated herein simply as “phage”) is a generic term for viruses that infect only bacteria. Many phages, which are also referred to as lytic phages, specifically adsorb to a target bacterium (host), which is a host, then inject their own DNA, and self-amplify using the translation mechanism of the bacterium. Further, the bacterium is lysed to disperse the amplified phage, and infection of other target bacterium is repeated (NPL 1).

Many bacteria of the genus Xanthomonas are known as causative bacteria of diseases of plants including various agricultural crops, and copper agents and antibiotics have been used for general conventional control. However, there are many problems such as drug efficacy, phytotoxicity, and possible causes of disruption of bacterial flora balance. Therefore, in recent years, a method using phage has been attracting attention as a new control method (NPL 2).

For example, PLTs 1 to 3 and NPL 2 report examples of phages having bacteriolytic activity against bacteria of the genus Xanthomonas. However, since the host range of phage is extremely narrow, it is still important to search for a novel phage or find a phage having higher bacteriolytic activity.

### CITATION LIST

### Patent Literature

PLT 1: Japanese Unexamined Patent Publication No. 2016-32435
PLT 2: Japanese Unexamined Patent Publication No. 2021-102635
PLT 3: International Publication No. 2017/113029

### Non Patent Literature

NPL 1: Sharma S. et al., Folia Microbiol., 2017, 62:17-55
NPL 2: Nakayinga R. et al., BMC Microbiology, 2021, 21:291

### SUMMARY

In order to solve the above-mentioned problems, the present inventors focused on bacteriophages. Unlike conventional copper agents and antibiotics, phages, which are viruses, are natural products, and therefore, manifestation of phytotoxicity has not been reported so far. In addition, since the specificity to the host is very high, only bacteria of a specific genus or species are targeted, and the influence on the bacterial flora balance is extremely limited. Further, phages are harmless not only to animals including humans but also to plants, and are highly safe. Therefore, in order to suppress damage to agricultural products due to plant diseases caused by bacteria of the genus Xanthomonas, a novel phage exhibiting bacteriolytic activity against bacteria of the genus Xanthomonas is isolated. Another objective of one or more embodiments of the present invention is to provide a composition containing the phage as an active ingredient, and to apply the composition to disease control, detection of pathogenic bacteria, and the like.

The present inventors isolated novel phages from natural sewage or soil by using a technique for detecting a lysis plaque formed on a soft agar culture medium on which a bacterium of the genus Xanthomonas has been cultured, and analyzed their genome sequences. As a result, it was revealed that the phages have novel genomic DNA sequences to which no analogous sequence has been known at all. One or more embodiments of the present invention has been completed based on the above research and development results. Specifically, the following are provided.
[1] A bacteriolytic agent comprising a bacteriophage having a genomic DNA sequence comprising a base sequence shown in any one of SEQ ID NOs: 1 to 3.
[2] The bacteriolytic agent according to [1], which exhibits bacteriolytic activity against a bacterium of the genus Xanthomonas.
[A-1] A bacteriolytic agent comprising a bacteriophage having a genomic DNA sequence comprising a base sequence represented by any one of the following (1a) to (1c): (1a) a base sequence shown in SEQ ID NO: 1, (1b) a base sequence shown in SEQ ID NO: 1 in which one or more bases are added, deleted, and/or substituted, and (1c) a base sequence having 95% or more sequence identity to the base sequence shown in SEQ ID NO: 1.
[A-2] The bacteriolytic agent according to [A-1], which exhibits bacteriolytic activity against a bacterium of the genus Xanthomonas.
[A-3] The bacteriolytic agent according to [A-2], wherein the bacterium of the genus Xanthomonas is at least one bacterium selected from the group consisting of Xanthomonas arboricola, Xanthomonas campestris and Xanthomonas citri.
[B-1] A bacteriolytic agent comprising a bacteriophage having a genomic DNA sequence comprising a base sequence represented by any one of the following (2a) to (2c) and having a full length of 40,000 bases or less: (2a) a base sequence shown in SEQ ID NO: 2, (2b) a base sequence shown in SEQ ID NO: 2 in which one or more bases are added, deleted, and/or substituted, or (2c) a base sequence having 90% or more sequence identity to the base sequence shown in SEQ ID NO: 2.
[B-2] The bacteriolytic agent according to [B-1], which exhibits bacteriolytic activity against a bacterium of the genus Xanthomonas.
[B-3] The bacteriolytic agent according to [B-2], wherein the bacterium of the genus Xanthomonas is Xanthomonas arboricola and/or Xanthomonas campestris.
[C-1] A bacteriolytic agent comprising a bacteriophage having a genomic DNA sequence comprising a base sequence represented by any one of the following (3a) to (3c): (3a) a base sequence shown in SEQ ID NO: 3, (3b) a base sequence shown in SEQ ID NO: 3 in which one or more bases are added, deleted, and/or substituted, or (3c) a base sequence having 90% or more sequence identity to the base sequence shown in SEQ ID NO: 3.
[C-2] The bacteriolytic agent according to [C-1], which exhibits bacteriolytic activity against a bacterium of the genus Xanthomonas.
[C-3] The bacteriolytic agent according to [C-2], wherein the bacterium of the genus Xanthomonas is Xanthomonas arboricola and/or Xanthomonas campestris.
[3] A composition comprising the bacteriolytic agent according to [1] or [2], or any one of [A-1] to [C-3] as an active ingredient.
[4] A plant disease control composition comprising the composition according to [3].
[5] The plant disease control composition according to [4], wherein the plant disease is a plant disease caused by a bacterium of the genus Xanthomonas.
[6] The plant disease control composition according to [4] or [5], which comprises another bacteriophage exhibiting bacteriolytic activity against a bacterium of the genus Xanthomonas.
[7] A method for controlling a plant disease, comprising a contact step of bringing the plant disease control composition according to any one of [4] to [6] into contact with a target plant.
[8] A method for identifying bacteria of the genus Xanthomonas, comprising: a culturing step of culturing test bacteria isolated from a plant tissue affected by a plant disease to obtain a culture; a mixing step of mixing the culture and the bacteriolytic agent according to [2] to obtain a mixture; a mixture-culturing step of culturing the mixture under a predetermined condition; and a determination step of determining that the test bacteria are bacteria of the genus Xanthomonas when the test bacteria are lysed after the mixture-culturing step.
[9] The method according to [8], wherein in the mixture-culturing step, the mixture further comprises a soft agar-containing liquid culture medium, and the mixture is cultured on a solid culture medium.
[10] The method according to [8], wherein in the culturing step, the culture comprises a soft agar-containing liquid culture medium, and the culture is cultured on a solid culture medium.
[11] The method according to any one of [8] to [10], further comprising an isolation step of isolating the test bacteria from the plant tissue affected by the plant disease prior to the culturing step.

This description includes the disclosure of Japanese Patent Application No. 2023-089380, based on which the present application claims priority.

### Advantageous Effects of One or More Embodiments of the Invention

The bacteriolytic agent of one or more embodiments of the present invention and a composition containing the bacteriolytic agent as an active ingredient can lyse a specific target bacterium.

According to the plant disease control composition of one or more embodiments of the present invention, diseases caused by specific target bacteria can be prevented and suppressed.

### BRIEF DESCRIPTION OF DRAWINGS

FIG. 1A shows the bacteriolytic activity of a first bacteriophage obtained in Example 1. FIG. 1A is a diagram showing the results of culturing bacteria of the genus Xanthomonas and Pseudomonas fluorescens for control on agar plates, then dropping a purified first phage liquid onto the center of each plate, and performing static culture.

FIG. 1B is a plate diagram corresponding to FIG. 1A, showing the bacteria shown in Table 2 spread on each plate.

FIG. 2A shows the bacteriolytic activity of the first bacteriophage obtained in Example 1. FIG. 2A is a diagram showing the results of culturing bacteria of the genus Xanthomonas and Pseudomonas fluorescens for control spread on agar plates, then dropping the purified first phage liquid onto the center of each plate, and performing static culture.

FIG. 2B is a plate diagram corresponding to FIG. 2A, showing the bacteria shown in Table 2 spread on each plate.

FIG. 3A shows the bacteriolytic activity of a second bacteriophage obtained in Example 2. FIG. 3A is a diagram showing the results of culturing bacteria of the genus Xanthomonas and Pseudomonas fluorescens for control on agar plates, dropping the purified second phage liquid onto the center of each plate, and performing static culture.

FIG. 3B is a plate diagram corresponding to FIG. 3A, showing the bacteria shown in Table 4 spread on each plate.

FIG. 4A shows the bacteriolytic activity of the second bacteriophage obtained in Example 2. FIG. 4A is a diagram showing the results of culturing bacteria of the genus Xanthomonas shown in Table 4 and Pseudomonas fluorescens for control spread on agar plates, and then dropping a purified second phage liquid to the center of each plate, and performing static culture.

FIG. 4B is a plate diagram corresponding to FIG. 4A, showing the bacteria shown in Table 4 spread on each plate.

FIG. 5A shows the bacteriolytic activity of a third bacteriophage obtained in Example 3. FIG. 5A is a diagram showing the results of culturing bacteria of the genus Xanthomonas and Pseudomonas fluorescens for control on agar plates, dropping a purified third phage liquid onto the central portion of each plate, and performing static culture.

FIG. 5B is a plate diagram corresponding to FIG. 5A, showing the bacteria shown in Table 5 spread on each plate.

FIG. 6A shows the bacteriolytic activity of combinations of the first bacteriophage with other bacteriophages tested in Example 4. FIG. 6A is a diagram showing the results of culturing bacteria of the genus Xanthomonas spread on agar plates, dropping a purified phage liquid on each plate, and performing static culture.

FIG. 6B is a plate diagram corresponding to FIG. 6A, and shows the types of phage contained in the purified phage liquid dropped on each plate. In FIGS. 6A and 6B, the two columns on the left (1, 2, 5, 6, 9 and 10) show the plates on which the bacteria whose identifier is MAFF No. 673005 were spread, and the two columns on the right (3, 4, 7, 8, 11 and 12) show the plates on which the bacteria whose identifier is MAFF No. 301352 were spread. In FIG. 6B, a represents the first phage having the genomic DNA sequence of SEQ ID NO: 1, b represents a phage having the genomic DNA sequence of SEQ ID NO: 4, c represents a phage having the genomic DNA sequence of SEQ ID NO: 5, d represents a phage having the genomic DNA sequence of SEQ ID NO: 6, e represents a phage having the genomic DNA sequence of SEQ ID NO: 7, f represents the second phage having the genomic DNA sequence of SEQ ID NO: 2, g represents the third phage having the genomic DNA sequence of SEQ ID NO: 3, and h represents a phage having the genomic DNA sequence of SEQ ID NO: 8. In the figure, “/” indicates that phages before and after “/” are used in combination. For example, “a/f” indicates that the first phage and the second phage are used in combination.

FIG. 7A shows the bacteriolytic activity of combinations of the second bacteriophage with other bacteriophages tested in Example 5. FIG. 7A is a diagram showing the results of culturing bacteria of the genus Xanthomonas spread on agar plates, dropping a purified phage liquid onto each plate, and performing static culture.

FIG. 7B is a plate diagram corresponding to FIG. 7A, and shows the types of phage contained in the purified phage liquid dropped on each plate. In FIGS. 7A and 7B, the two columns on the left (1, 2, 5, 6, 9 and 10) show the plates on which bacteria whose identifier is MAFF No. 673005 were spread, and the two columns on the right (3, 4, 7, 8, 11 and 12) show the plates on which bacteria whose identifier is MAFF No. 301352 were spread. In FIG. 7B, a represents the second phage having the genomic DNA sequence of SEQ ID NO: 2, b represents the phage having the genomic DNA sequence of SEQ ID NO: 4, c represents the phage having the genomic DNA sequence of SEQ ID NO: 5, d represents the phage having the genomic DNA sequence of SEQ ID NO: 6, e represents the phage having the genomic DNA sequence of SEQ ID NO: 7, f represents the first phage having the genomic DNA sequence of SEQ ID NO: 1, g represents the third phage having the genomic DNA sequence of SEQ ID NO: 3, and h represents the phage having the genomic DNA sequence of SEQ ID NO: 8. In the figure, “/” indicates that phages before and after “/” are used in combination. For example, “a/f” indicates that the second phage and the first phage are used in combination.

FIG. 8A shows the bacteriolytic activity of combinations of the second bacteriophage with other bacteriophages tested in Example 6. FIG. 8A is a diagram showing the results of culturing bacteria of the genus Xanthomonas spread on agar plates, dropping a purified phage liquid onto each plate, and performing static culture.

FIG. 8B is a plate diagram corresponding to FIG. 8A, and shows the types of phage contained in the purified phage liquid dropped on each plate. In FIGS. 8A and 8B, the left two columns (1, 2, 5, 6, 9, and 10) show plates on which bacteria having an identifier of MAFF No. 673005 were spread, the third column from the left (3, 7, and 11) shows plates on which bacteria having an identifier of MAFF No. 301257 were spread, and the rightmost column (4, 8, and 12) shows plates on which bacteria having an identifier of MAFF No. 106765 were spread. In FIG. 8B, a represents the third phage having the genomic DNA sequence of SEQ ID NO: 3, b represents the phage having the genomic DNA sequence of SEQ ID NO: 4, c represents the phage having the genomic DNA sequence of SEQ ID NO: 5, d represents the phage having the genomic DNA sequence of SEQ ID NO: 6, e represents the phage having the genomic DNA sequence of SEQ ID NO: 7, f represents the first phage having the genomic DNA sequence of SEQ ID NO: 1, g represents the second phage having the genomic DNA sequence of SEQ ID NO: 2, and h represents the phage having the genomic DNA sequence of SEQ ID NO: 8. In the figure, “/” indicates that phages before and after “/” are used in combination. For example, “a/f” indicates that the third phage and the first phage are used in combination.

FIG. 9 is a graph showing the results of a test for disease control effect on tomato bacterial blight tested in Example 7. The average incidence rate is given as a relative value to the untreated group.

FIG. 10 is a graph showing the results of a test for disease control effect on broccoli black rot tested in Example 8. The average incidence rate is given as a relative value to the untreated group. The numerical value shown in each bar graph indicates the type of phage used. In the figure, a represents the third phage having the genomic DNA sequence of SEQ ID NO: 3, b represents the phage having the genomic DNA sequence of SEQ ID NO: 9, c represents the phage having the genomic DNA sequence of SEQ ID NO: 8, d represents the phage having the genomic DNA sequence of SEQ ID NO: 10, e represents the phage having the genomic DNA sequence of SEQ ID NO: 5, f represents the phage having the genomic DNA sequence of SEQ ID NO: 6, g represents the phage having the genomic DNA sequence of SEQ ID NO: 7, and h represents the phage having the genomic DNA sequence of SEQ ID NO: 11. In the figure, “/” indicates that phages before and after “/” are used in combination. For example, “a/b” indicates that the third phage and the phage having the genomic DNA sequence of SEQ ID NO: 9 are used in combination.

### DESCRIPTION OF EMBODIMENTS

### 1. Bacteriolytic Agent

### 1-1. Overview

A first aspect of one or more embodiments of the present invention is a bacteriolytic agent. The bacteriolytic agent comprises a bacteriophage having a genome sequence containing a specific base sequence.

The bacteriolytic agent of one or more embodiments of the present invention exhibits bacteriolytic activity specific to a target bacterium which can be a pathogenic bacterium of a plant disease.

### 1-2. Definition

The terms used herein are defined below.

As used herein, the term “bacteriolytic agent” refers to an agent comprising a bacteriophage having bacteriolytic activity against a target bacterium.

“Bacteria” are one of the major lineages of organisms that, together with archaea and eukaryotes, trisect the entire kingdom of life. Bacteria are made of cells without a cell nucleus and are capable of self-replication if there is a nutrient source. The names of bacteria are indicated by genus and species under the family based on the International Code of Nomenclature of Bacteria.

As used herein, the term “target bacterium” refers to a host bacterium which can be a target of the phage constituting the bacteriolytic agent of one or more embodiments of the present invention or the phage contained in the composition and the plant disease control composition of one or more embodiments of the present invention. For example, the target bacterium is a bacterium having a membrane surface receptor on the outer cell membrane that is recognized by the phage. The “membrane surface receptor” is a site to which, for example, a tail and a tail fiber of a phage bind, and is composed of a protein, a lipopolysaccharide, a pilus, and the like present in the outer layer of a bacterial outer membrane. Specific examples of the target bacteria in the present specification include bacteria of the genus Xanthomonas.

The “bacteria of the genus Xanthomonas” are bacteria belonging to the genus Xanthomonas. Bacteria of the genus Xanthomonas generally produce a yellow pigment called xanthomonadin, many of which are known as plant pathogenic bacteria. Further, there are subtypes and pathovars as a lower classification than the species, and they are indicated by adding subsp. or pv. after the bacterial name. The minimum unit of classification is a strain, which refers to a population of cells considered to be genetically uniform. The following Table 1 shows representative bacteria of the genus Xanthomonas, host plants thereof, and plant diseases caused thereby.

TABLE 1

| Species | Pathovar | Host | Disease |
|---|---|---|---|
| X. arboricola | pruni | Peach | Bacterial spot |
| X. arboricola | juglandis | Walnut | Bacterial blight |
| X. arboricola | corylina | Turkish Hazel | Leaf spot |
| X. axonopodis | citrumelo | Citrus | Bacterial spot |
| X. axonopodis | glycines | Soybean | Bacterial pustule |
| X. axonopodis | manihotis | Cassava | Bacterial blight |
| X. axonopodis | malvacearum | Cotton | Bacterial blight |
| X. axonopodis | punicae | Pomegranate | Leaf blight |
| X. albilineans | | Sugarcane | Leaf scald |
| X. campestris | campestris | Cabbage | Black rot |
| X. campestris | musacearum | Banana | Bacterial wilt |
| X. campestris | vasculorum | Sugarcane | Gumming disease |
| X. campestris | vesicatoria | Tomate/Pepper | Bacterial spot |
| X. campestris | vitians | Lettuce | Bacterial spot |
| X. campestris | raphani | Cabbage | Leaf spot |
| X. citri | citri | Orange | Bacterial canker |
| X. citri | malvacearum | Cotton | Angular leaf spot |
| X. citri | mangiferaeindicae | Mango | Black spot |
| X. cucurbitae | | Pumpkin | Bacterial Spot |
| X. fragariae | | Strawberry | Angular leaf spot |
| X. fuscans | fuscans | Bean | Bacterial blight |
| X. hortorum | carotae | Carrot | Bacterial blight |
| X. oryzae | oryzae | Rice | Leaf blight |

Without limitation, among the bacteria of the genus Xanthomonas, Xanthomonas arboricola, Xanthomonas citri and Xanthomonas campestris are particularly preferred as the target bacteria of one or more embodiments of the present invention. Specific examples of Xanthomonas arboricola include Xanthomonas arboricola pv. pruni, the pathovar of which is pruni, and Xanthomonas arboricola pv. juglandis, the pathovar of which is juglandis. Xanthomonas citri subsp. citri may be mentioned as a specific example of Xanthomonas citrifolia. Specific examples of Xanthomonas campestris include Xanthomonas campestris pv. vesicatoria, the pathovar of which is vesicatoria, Xanthomonas campestris pv. vitians, the pathovar of which is vitians, and Xanthomonas campestris pv. campestris, the pathovar of which is campestris.

“Bacteriophage” (as mentioned above, often abbreviated herein simply as “phage”) is a generic term for viruses that infect bacteria. A general phage is composed of three parts, i.e., a head, a tail, and a tail fiber. The head is composed of a capsid (virus shell) having an icosahedral structure composed of a capsomere which is an outer coat protein, and contains the genomic DNA of the phage in the inner space thereof. The tail has a tubular structure composed of a tail tube protein and a sheath protein covering the tail tube protein. One end of the tail is connected to the head and the other end to the tail fiber. The tail serves as a transfer tube through which the genomic DNA in the head is injected into a bacterial host cell. The tail and tail fiber are composed of several fiber structures composed of tail fiber proteins. The tail and tail fiber are responsible for host recognition and adsorption functions, recognizing receptors present on the outer membrane surface of the host bacterium and adsorbing to its cell surface. Phages are highly host-specific, a feature based on the function of the tail and tail fiber. Since phages do not infect eukaryotes, agents using phages are harmless to humans, animals, and plants. Phages are roughly classified into “lytic cycle”, “lysogenic cycle”, and “lytic/lysogenic cycle” based on the mode of infection. In the lysogenic cycle, the phage integrates its own DNA into the bacterial chromosome without lysing the target bacterium and multiplies with bacterial growth. On the other hand, in the lytic cycle, the phage self-multiplies in the cell of the host bacterium, and then lyses the host bacterium to release a large amount of progeny phages. The phage of one or more embodiments of the present invention is a virulent phage that undergoes a lytic cycle.

As described above, the “tail fiber protein” is a protein constituting a tail fiber of a phage. The tail fiber protein is known to play important roles in the specificity of the host recognition and adsorption capability of the tail and tail fiber (Nobrega F. L. et al., Nat. Rev. Microbiol., 2018, 16:760-773).

The term “tail fiber gene” refers to a gene that is contained in the genomic DNA of a phage and encodes the tail fiber protein.

As described above, the “tail tube protein” is a protein constituting the tubular structure of the tail of a phage. The tail tube protein is known to interact with the tail fiber and, with the tail-fibers, play important roles in specificity of host recognition and adsorption capability (Maozhi Hu, et ai., 2020, 9:1, 855-867). As tail tube proteins, tail tube fiber protein A and tail tube protein B are known. The term “tail tube protein A” is a protein that forms a ring at a lower portion of the tubular structure of the tail and interacts with the tail fiber. The term “tail tube protein B” is a protein that forms the lower end of the tubular structure of the tail and binds to a receptor present on the outer membrane surface of the host bacterium.

The term “tail tube gene” refers to a gene that is contained in the genomic DNA of a phage and encodes the tail tube protein. The term “tail tube protein A gene” refers to a gene encoding tail tube protein A, and the term “tail tube protein B gene” refers to a gene encoding tail tube protein B.

“(Bacterio) lysis” refers to the phenomenon of destroying the cell membrane of a bacterium. As described above, this is a phenomenon mainly observed in the infection mode of virulent phage. By lysis, the bacteria are killed. Lysis starts with the phage specifically adsorbing to the target bacterium and injecting its DNA into the cell of the target bacterium via the tail. Thereafter, the phage replicates itself by utilizing the translation mechanism of the bacterium to produce a large amount of progeny phages, and then lyses the bacterium to release the progeny phages to the outside.

As used herein, the term “plant disease” is a generic term for diseases that occur in plants. Known plant diseases include diseases caused by infectious pathogens such as viruses, bacteria, filamentous fungi, actinomycetes, viroids, phytoplasmas, nematodes, mites, and insects, and diseases caused by non-infectious pathogens such as deficiency or excess of nutrients or water, and phytotoxicity. Plant diseases in the present specification refer to diseases caused by bacteria, i.e., plant pathogenic bacteria, unless otherwise specified. In the present specification, the plant pathogenic bacteria correspond to the above-described target bacteria, for example, bacteria of the genus Xanthomonas, unless otherwise specified.

As used herein, the term “control” refers to prevention or treatment (extermination) (according to HP of Japan Crop Protection Association). Therefore, the term “plant disease control” as used herein refers to the prevention of plant diseases, particularly against target bacteria, or the treatment of plant diseases caused by target bacteria.

As used herein, the term “target plant” refers to a plant to which the plant disease control composition of one or more embodiments of the present invention described below is applied. This plant corresponds to a plant which develops a specific plant disease due to infection with the target bacterium, or a plant which may be infected with the target bacterium.

### 1-3. Constitution

The bacteriolytic agent of one or more embodiments of the present invention comprises a bacteriophage.

### <First Phage>

The first phage is characterized by having a genomic DNA sequence comprising a specific base sequence, and the bacteriolytic agent of one or more embodiments of the present invention exhibits bacteriolytic activity specific to a target bacterium.

### (1-1) Genomic DNA

The first phage is such that the DNA sequence of the genome of the phage has the base sequence of 42,933 bp shown in SEQ ID NO: 1, or a base sequence shown in SEQ ID NO: 1 in which one or more bases are added, deleted and/or substituted, or a base sequence having a sequence identity of 92.15% or more, 92.2% or more, 92.3% or more, 92.5% or more, 93% or more, 93.5% or more, 94% or more, 94.5% or more, 95% or more, 95.5% or more, 96% or more, 96.5% or more, 97% or more, 97.5% or more, 98% or more, 98.5% or more, 99% or more, or 99.5% or more to the base sequence shown in SEQ ID NO: 1, or a base sequence having a sequence identity of 99.01% or more, 99.05% or more, 99.1% or more, 99.2% or more, 99.3% or more, 99.4% or more, 99.5% or more, 99.6% or more, 99.7% or more, 99.8% or more, or 99.9% or more when aligned with the base sequence shown in SEQ ID NO: 1, furthermore, a base sequence which hybridizes to a base sequence complementary to the base sequence shown in SEQ ID NO: 1 under highly stringent conditions.

It is not easy to align the entire range of a long genomic DNA sequence and use the aligned range as a comparison range. The range to be compared (hereinafter, sometimes referred to as Query Cover) is most particularly 100% of the entire genome of the first phage, but may be 99% or more, 98% or more, 97% or more, 96% or more, 95% or more, 94% or more, 93.5% or more, or 93% or more.

As used herein, the term “a plurality of” refers to 2 to 10, for example, 2 to 7, 2 to 5, 2 to 4, or 2 to 3.

As used herein, the term “(base) sequence identity” is a numerical value indicating the proportion of sites where the types of bases are identical within the comparison range between two base sequences. Even when two base sequences have different lengths, the base sequence identity can be calculated by aligning the two base sequences so that the degree of base identity within the comparison range is maximized. An exemplary, non-limiting algorithm for performing such an analysis is BLAST. BLAST can be used in various software and Web services. For example, the base sequence identity can be easily calculated using genetic information processing software GENETYX (https://www.genetyx.co.jp/), BLAST server (https://blast.ncbi.nlm.nih.gov/Blast.cgi) provided by NCBI, or the like. In addition to BLAST, there is an algorithm called FASTA or the like, which can be used as long as an appropriate identity can be calculated, and the method used is not particularly limited.

The term “highly stringent conditions” refers to environmental conditions under which non-specific hybridization is unlikely to occur. Under highly stringent conditions, a hybrid can be formed with a nucleic acid having a target base sequence, but a hybrid cannot be substantially formed with a nucleic acid having a nonspecific base sequence. In general, highly stringent conditions refer to conditions of low salt concentration and high temperature. The low salt concentration refers to, for example, 15 mM to 750 mM, particularly 15 mM to 500 mM, 15 mM to 300 mM or 15 mM to 200 mM. The high temperature is, for example, 50 to 68° C. or 55 to 70° C. Specific examples of the highly stringent conditions include conditions of washing at 65° C., 0.1×SSC and 0.1% SDS in washing after hybridization.

From another viewpoint, the phage is a virus, and there is a possibility that a mutation such as substitution, deletion, or insertion may occur in the genomic DNA when the phage of one or more embodiments of the present invention is amplified. As long as the degree of the mutation is within the above-described range with respect to the entire genomic DNA and the lytic function of the phage is maintained, the phage is included in one or more embodiments of the present invention even with such a mutation.

Depending on the software or the analysis server, Average Nucleotide Identity (ANI) or the like may be used as an index indicating sequence identity. It should be noted that it is not easy to align and arrange a long phage genomic DNA to cover the entire range to be compared. Therefore, the above-mentioned sequence identity may be present in the range of automatic alignment and arrangement by the above-mentioned software or Web service. For example, by analysis using the BLAST server provided by NCBI, a query sequence and a subject sequence are automatically aligned (herein often referred to as “aligned arrangement”) in the maximum alignable range, and a comparison range is determined. The sequence identity in the comparison range is calculated, and the ratio of the comparison range to the entire range of the query sequence may be calculated as a value called Query Cover.

These values may vary depending on how the query sequence and the subject sequence are selected. This point will be briefly described by taking a first sequence composed of a sequence A and a second sequence composed of the sequence A and a sequence B having the same length as the sequence A as an example. When the shorter first sequence is the query sequence and the longer second sequence is the subject sequence, the comparison range is the sequence A, and since the sequence A is the entire length of the first sequence, the calculated value of Query Cover is 100%. In addition, since the sequence A in the first sequence is the same as that in the second sequence, the value of sequence identity is 100%. On the other hand, when the longer second sequence is used as the query sequence and the shorter first sequence is used as the subject sequence, the value of the sequence identity is 100% as described above, but the value of the Query Cover is 50% because the sequence A occupies only half of the second sequence.

In such a case, the sequence identity of the base sequence in the entire range of the aligned base sequences (sequence identity in the entire range) can be estimated based on the result. For example, the value obtained by multiplying the Query Cover value by the value of sequence identity in the aligned and compared range (sequence identity in the aligned range) can be used as the estimated value of sequence identity in the entire range. At this time, in order to increase the accuracy of the estimated value, for example, a further correction such as inclusion of an expected sequence identity in a range other than the aligned range may be made.

The genomic DNA of the phage may be packaged in a linear form or in a circular form. In the next-generation genome sequencer analysis, genomic DNA is fragmented, then the base sequence of each fragment is read, and the sequence is determined through an analysis for connecting the fragments. In the case of phage, they are often connected without a reference genomic DNA sequence (de novo assembly). Therefore, it is difficult to unambiguously determine the starting point and end of the analyzed genome (Merrill, B. D., et al. BMC Genomics, 2016 17, 679). The starting points and the ends of the genome sequences to be compared may be different, and are automatically considered in the analysis using software or an analysis server.

Although not particularly limited, among the bacteria of the genus Xanthomonas, Xanthomonas arboricola, Xanthomonas citri and Xanthomonas campestris are particularly preferred as target bacteria of the first phage. Specific examples of Xanthomonas arboricola suitable as target bacteria of the first phage include Xanthomonas arboricola pv. pruni, the pathovar of which is pruni, and Xanthomonas arboricola pv. juglandis, the pathovar of which is juglandis. A specific example of Xanthomonas citri suitable as a target bacterium of the first phage is Xanthomonas citri subsp. citri. Specific examples of Xanthomonas campestris suitable as target bacteria of the first phage include Xanthomonas campestris pv. vesicatoria, the pathovar of which is vesicatoria, and Xanthomonas campestris pv. vitians, the pathovar of which is vitians.

### (1-2) Effect

The bacteriolytic agent of one or more embodiments of the present invention comprising the first phage can exhibit bacteriolytic activity against bacteria of the genus Xanthomonas and can be applied to plant diseases.

### <Second Phage>

The second phage is characterized by having a genomic DNA sequence of a specific length comprising a specific base sequence, and the bacteriolytic agent of one or more embodiments of the present invention exhibits bacteriolytic activity specific to a target bacterium.

### (2-1) Genomic DNA

The second phage is such that the DNA sequence of the genome of the phage has a base sequence of 35,321 bp shown in SEQ ID NO: 2, or a base sequence shown in SEQ ID NO: 2 in which one or more bases are added, deleted and/or substituted, or a base sequence having a sequence identity of 90% or more, 90.5% or more, 91% or more, 91.5% or more, 92% or more, 92.5% or more, 93% or more, 93.5% or more, 94% or more, 94.5% or more, 95% or more, 95.5% or more, 96% or more, 96.5% or more, 97% or more, 97.5% or more, 98% or more, 98.5% or more, 99% or more or 99.5% or more to the base sequence shown in SEQ ID NO: 2, a base sequence having a sequence identity of 95.0% or more, 95.5% or more, 96.0% or more, 96.5% or more, 97.0% or more, 97.5% or more, 98.0% or more, 98.5% or more, 99% or more, 99.5% or more, 99.6% or more, 99.7% or more, 99.8% or more, or 99.9% or more when aligned with the base sequence shown in SEQ ID NO: 2, furthermore, a base sequence that hybridizes to a base sequence complementary to the base sequence shown in SEQ ID NO: 2 under highly stringent conditions.

It is not easy to use the entire range of a long genomic DNA sequence as a comparison range. The range to be compared (hereinafter may be referred to as Query Cover) is most particularly 100% of the entire genome length of the second phage, but may be 99% or more, 98% or more, 97% or more, 96% or more, 95% or more, 94% or more, or 93% or more.

The genomic DNA sequence of the second phage has a total length of 40,000 bases or less. For example, the full length of the genomic DNA sequence is 40,000 bases or less, 39,500 bases or less, 39,000 bases or less, 38,500 bases or less, 38,000 bases or less, 37,500 bases or less, 37,000 bases or less, 36,500 bases or less, 36,000 bases or less, 35,900 bases or less, 35,800 bases or less, 35,700 bases or less, 35,600 bases or less, 35,500 bases or less, 35,400 bases or less.

Although not particularly limited, among the bacteria of the genus Xanthomonas described above in the definition section, Xanthomonas arboricola and Xanthomonas campestris are particularly preferred as the target bacteria of the second phage. Specific examples of Xanthomonas arboricola suitable as target bacteria for the second phage include Xanthomonas arboricola pv. pruni, the pathovar of which is pruni, and Xanthomonas arboricola pv. juglandis, the pathovar of which is juglandis. Specific examples of Xanthomonas campestris suitable as target bacteria of the second phage include Xanthomonas campestris pv. vesicatoria, the pathovar of which is vesicatoria, Xanthomonas campestris pv. vitians, the pathovar of which is vitians, and Xanthomonas campestris pv. campestris, the pathovar of which is campestris.

### (2-2) Effect

The bacteriolytic agent of one or more embodiments of the present invention containing the second phage can exhibit bacteriolytic activity against bacteria of the genus Xanthomonas, and can be applied to plant diseases.

### <Third Phage>

The third phage is characterized by having a genomic DNA sequence containing a specific base sequence, and the bacteriolytic agent of one or more embodiments of the present invention exhibits bacteriolytic activity specific to a target bacterium.

### (3-1) Genomic DNA

The third phage is such that the DNA sequence of the genome of the phage has the base sequence of 43,601 bp shown in SEQ ID NO: 3, or a base sequence shown in SEQ ID NO: 3 in which one or more bases are added, deleted and/or substituted, or a base sequence having a sequence identity of 90% or more, 90.5% or more, 91% or more, 91.5% or more, 92% or more, 92.5% or more, 93% or more, 93.5% or more, 94% or more, 94.5% or more, 95% or more, 95.5% or more, 96% or more, 96.5% or more, 97% or more, 97.5% or more, 98% or more, 98.5% or more, 99% or more, or 99.5% or more to the base sequence shown in SEQ ID NO: 3, or a base sequence having a sequence identity of 96.9% or more, 97.0% or more, 97.5% or more, 98.0% or more, 98.5% or more, 99% or more, 99.5% or more, 99.6% or more, 99.7% or more, 99.8% or more, 99.9% or more when aligned with the base sequence shown in SEQ ID NO: 3, furthermore, a base sequence that hybridizes to a base sequence complementary to the base sequence shown in SEQ ID NO: 3 under highly stringent conditions.

It is not easy to use the entire range of a long genomic DNA sequence as a comparison range. The range to be compared (hereinafter, sometimes referred to as Query Cover) is most particularly 100% of the entire genome length of the third phage, but may be 99% or more, 98% or more, 97% or more, 96% or more, 95% or more, 94% or more, 93% or more, 92% or more, 91% or more, 90% or more, 89% or more, or 88% or more.

Although not particularly limited, among the bacteria of the genus Xanthomonas described above in the definition section, Xanthomonas arboricola and Xanthomonas campestris are desirable as target bacteria of the third phage. A specific example of Xanthomonas arboricola suitable as a target bacterium of the third phage may be Xanthomonas arboricola pv. juglandis, the pathovar of which is juglandis. Specific examples of Xanthomonas campestris suitable as target bacteria of the third phage include Xanthomonas campestris pv. vesicatoria, the pathovar of which is vesicatoria, Xanthomonas campestris pv. vitians, the pathovar of which is vitians, and Xanthomonas campestris pv. campestris, the pathovar of which is campestris.

### (3-2) Effect

The bacteriolytic agent of one or more embodiments of the present invention can exhibit bacteriolytic activity against bacteria of the genus Xanthomonas and can be applied to plant diseases.

### 2. Plant Disease Control Composition

### 2-1. Overview

A second aspect of one or more embodiments of the present invention is a composition, particularly a composition which can be used for controlling a plant disease. The composition of one or more embodiments of the present invention is characterized by comprising the bacteriolytic agent according to the first aspect as an active ingredient.

According to the composition of one or more embodiments of the present invention, it is possible to provide a sustainable agricultural agent against bacterial plant diseases, which is safe for the human body, has no drug-induced harmful effect on the environment, and is capable of specifically preventing or treating a target plant disease, when used for controlling a plant disease.

In the present specification, the term “plant disease control composition” refers to a case where the composition of one or more embodiments of the present invention is used for plant disease control.

### 2-2. Constitution

### 2-2-1. Components

The composition of one or more embodiments of the present invention comprises, as an essential component, the bacteriophage which is the bacteriolytic agent according to the first aspect as an active ingredient. An agriculturally acceptable carrier and/or medium may be included as long as it does not inhibit or suppress the bacteriolytic activity of the phage against the target bacteria. If necessary, the composition may further comprise other active ingredients. Hereinafter, each component will be described in detail.

### (1) Active Ingredient (Bacteriolytic Agent)

The composition of one or more embodiments of the present invention comprises the bacteriolytic agent according to the first aspect as an essential active ingredient. In the plant disease control composition, the target bacteria of one or more embodiments of the present invention are lysed by the active ingredient, and thus plant diseases caused by the target bacteria can be prevented or treated.

Since the specific constitution of the bacteriolytic agent has been described in detail in the first aspect, the description thereof is omitted here.

When the composition is used for controlling a plant disease, the amount of the active ingredient contained per unit amount in the composition varies depending on various conditions such as the dosage form, the type of plant pathogenic bacteria, the type of target plant, the place of application, and the method of application. It is desirable that the phage as an active ingredient is contained in an amount sufficient for contacting and infecting a plant pathogenic bacteria that have infected the target plant. Therefore, the amount of the bacteriolytic agent contained in the plant disease control composition of one or more embodiments of the present invention may be determined in consideration of each condition so that the bacteriolytic agent is contained in an effective amount against the target bacteria after application within the scope of common technical knowledge in the above-mentioned field.

### (2) Agriculturally Acceptable Carriers and Media

The term “agriculturally acceptable carrier and/or medium” refers to a substance that facilitates the application of the composition, can maintain the viability and infectivity of the phage as an active ingredient, and/or can control the rate of action, and has no or very little harmful influence on the environment such as soil and water quality when applied outdoors, and further has no or very little harmful influence on animals, particularly humans.

### (2-1) Carriers

Specific examples of agriculturally acceptable carriers include surfactants, protectants, excipients, and the like. If desired, minor amounts of wetting agents, emulsifying agents, pH buffering agents and the like may be utilized. The carriers may be pre-formulated or may be formulated just prior to application.

The surfactant has an effect of improving the physicochemical properties of the composition for plants, such as wettability, emulsifiability, dispersibility, penetrability, adhesiveness, defoaming property, and spreadability. The surfactant can be used as a main component of an agricultural adjuvant called a spreader. Examples of the spreader include a nonionic surfactant, a combination of a nonionic surfactant and an anionic surfactant, a paraffin-based compound, a paroxyethylene resin acid ester, and the like. More specific examples thereof include a polyoxyethylene alkyl ether compound, a polyoxyethylene fatty acid ester compound, a lignosulfonate compound, a naphthylmethanesulfonate compound, an alkylsulfosuccinate compound, and a tetraalkylammonium salt compound.

In the case of phage, the protectant is expected to have an effect of reducing damage caused by ultraviolet rays and the like. Examples thereof include skim milk, casein, gelatin and the like.

Examples of the excipient include glucose; lactose; sucrose; gelatin; starch; malt, wheat flour, and the like.

### (2-2) Solvent

Specific examples of agriculturally acceptable solvents include water (including aqueous solutions), buffers, or liquid culture media. The solvent is particularly a sterile liquid.

### (3) Other Active Ingredients

The composition of one or more embodiments of the present invention can comprise, in addition to the bacteriolytic agent described in the first aspect, one or more other active ingredients having the same and/or different pharmacological effect within a range where the bacteriolytic activity of the phage constituting the bacteriolytic agent is not affected.

The kind of other active ingredients is not limited. For example, it may be a phage having bacteriolytic activity against the same and/or different bacteria. Examples of the phage having bacteriolytic activity against the same bacterium include other phages which specifically recognize and bind to a bacterium of the genus Xanthomonas, similarly to the phage constituting the bacteriolytic agent described in the first aspect. For example, even when the target bacterium is the same, if the phages recognize different cell surface receptors, a synergistic effect or a complementary effect of the bacteriolytic activity can be expected by the combination thereof.

There are no particular limitations on the type of such other phage that specifically recognizes bacteria of the genus Xanthomonas and has bacteriolytic activity. Specific examples thereof include a phage whose genomic DNA comprises a base sequence shown in any one of SEQ ID NOs: 4 to 11, a mutant thereof, and a variant thereof.

The plant disease control composition of one or more embodiments of the present invention can comprise, as active ingredients, one or more other phages specifically recognizing bacteria of the genus Xanthomonas and having bacteriolytic activity as specifically exemplified above in combination, in addition to the bacteriolytic agent described in the first aspect.

In the above, the sequence identity of the amino acid sequence is particularly 95% or more, 96% or more, 97% or more, 98% or more, or 99% or more. As described above, the term “a plurality of” as used herein means 2 to 10, for example, 2 to 7, 2 to 5, 2 to 4, or 2 to 3.

The term “(amino acid) substitution” refers to a substitution between 20 amino acids constituting natural proteins within the corresponding one of conservative amino acid groups having similar properties such as charge, side chain, polarity, aromaticity, or the like. Examples of the substitution include a substitution within the group of uncharged polar amino acids having less polar side chains (Gly, Asn, Gln, Ser, Thr, Cys, Tyr), a substitution within the branched chain amino acid group (Leu, Val, Ile), a substitution within the neutral amino acid group (Gly, Ile, Val, Leu, Ala, Met, Pro), a substitution within the neutral amino acid group having hydrophilic side chains (Asn, Gln, Thr, Ser, Tyr, Cys), a substitution within the acidic amino acid group (Asp, Glu), a substitution within the basic amino acid group (Arg, Lys, His), and a substitution within the aromatic amino acid group (Phe, Tyr, Trp). Amino acid substitutions within these groups are particularly used because they are known to be less likely to alter the properties of the polypeptide.

Furthermore, in the present specification, “amino acid sequence identity” is a numerical value indicating the proportion of sites where the types of amino acid residues are identical within the comparison range of two amino acid sequences. The amino acid sequence identity can be calculated by aligning two amino acid sequences so that the degree of amino acid identity within the comparison range is the highest even when the two amino acid sequences have different lengths. An algorithm used for performing such an analysis can be, but is not limited to, the algorithm described above, such as BLAST.

In addition, insecticides, herbicides, fertilizers (for example, urea, ammonium nitrate, and superphosphate), and, if necessary, known agricultural chemicals, antibiotics, and agricultural biologicals can be included as other active ingredients.

### 2-2-2. Dosage Forms

When the composition of one or more embodiments of the present invention is used as a plant disease control composition, the composition may be in any dosage form as long as it can maintain the site of infection of a target bacterium on a target plant, the ability to colonize the target plant, and/or the ability of the phage as an active ingredient to easily infect the target bacterium. For example, the plant disease control composition may be suspended in an appropriate solution to form a liquid formulation or a wettable powder in a liquid state, or may be mixed with a carrier and solidified to form a powder, a granule, or a gel in a solid state. For example, when the infection site of the target bacterium in the target plant is a leaf, a flower, a fruit, a stem, a branch, or a trunk of the above-ground part, a liquid formulation, a wettable powder, or a gel, which is widely spread to the infection site and has high colonizing property, is suitable, but not limited thereto. On the other hand, when the infection site of the target bacterium is a root of an underground part or an underground stem, a powder or granule which is sustainedly released in the soil and can continuously exert an effect on the infection site is suitable, but not limited thereto.

### 2-3. Application Method

When the composition of one or more embodiments of the present invention is used as a plant disease control composition, the method for applying the composition is not particularly limited, and any method known in the art may be used as long as it can apply the plant disease control composition to a target plant. The phage, which is an active ingredient of the plant disease control composition, can be applied by an appropriate method according to the purpose because the phage can invade the target plant through the entire plant body surface such as the foliage part and root part thereof. For example, when the application site is an above-ground part such as a foliage part, the plant disease control composition may be applied so as to come into direct contact with the application site. Examples of the direct contact include application, spraying, sprinkling, and immersion of the plant disease control composition to the application site. It is particularly desirable that the application is to a site of infection or at risk of infection of target plant with the target bacteria. Further, when the application site is an underground portion such as a root portion, it may be indirectly applied by being added to soil, or in a case of a culture medium, to the culture medium. The term “soil” used herein is not particularly limited as long as it is a soil in which a target plant can grow. Normally, a planting soil containing appropriate nutrients and having an appropriate pH value is utilized. The location of the soil is not critical. Further, the term “culture medium” refers to an artificially prepared culture medium for planting a target plant. The culture medium may be a solid culture medium such as an agar culture medium or may be a liquid culture medium. Examples of culture media include, for example, isolated beds, root-zone restricted pots or nursery beds. The composition of the culture medium may be a culture medium composition known in the art. It can be appropriately selected depending on the kind of plant and the like.

### 2-4. Target Plant

The type of the target plant of the plant disease control composition of one or more embodiments of the present invention is not particularly limited as long as it is a plant which may develop a plant disease caused by the target bacterium of one or more embodiments of the present invention. The target plant may be either an angiosperm or a gymnosperm. Further, the target plant may be a herbaceous plant or a woody plant. Particular examples of the target plant include agriculturally important plants, for example, crop plants such as grains, vegetables, and fruits, and floricultural plants. Specific examples include monocotyledons such as Poaceae plants (for example, rice, wheat, barley, corn, sugarcane, sorghum, kaoliang, turfgrass), Musaceae plants (for example, banana), Amaryllidaceae plants (for example, Welsh onion, common onion, garlic, garlic chives), Liliaceae plants (for example, lily, tulip). Also included are dicotyledons such as Brassicaceae plants (for example, cabbage, radish, napa cabbage, rapeseed), Asteraceae plants (for example, lettuce, burdock, chrysanthemum), Fabaceae plants (for example, soybean, peanut, pea, bean, lentil, chickpea, faba bean, licorice), Solanaceae plants (for example, tomato, eggplant, potato, tobacco, bell pepper, capsicum, and petunia), Rosaceae plants (for example, strawberry, apple, pear, peach, loquat, almond, plum, rose, Japanese apricot, and cherry), Cucurbitaceae plants (for example, cucumber, cucurbits, pumpkin, melon, and watermelon), Anacardiaceae plants (for example, mango, pistachio, and cashew nut), Lauraceae plants (for example, avocado), Rutaceae plants (for example, mandarin orange, grapefruit, lemon, and yuzu), Convolvulaceae plants (for example, sweet potato), Theaceae plants (for example, tea plant), and Vitaceae plants (for example, grape).

### 2-5. Target Plant Diseases

The target plant diseases to which the plant disease control composition of one or more embodiments of the present invention is applied include all plant diseases caused by the target bacteria of one or more embodiments of the present invention. Plant diseases caused by bacteria of the genus Xanthomonas are particularly targeted. Examples thereof include bacterial spot found in peaches and the like, bacterial blight found in walnuts and the like, bacterial pustule found in soybeans and the like, angular leaf spot found in strawberries and the like, bacterial blight found in tomato, bell peppers, lettuce and the like), black rot found in cabbage, napa cabbage, broccoli and the like), bacterial canker found in oranges grapefruits and the like), angular leaf spot found in cotton and the like), leaf blight found in rice, and the like.

### 3. Method for Controlling Plant Disease

### 3-1. Overview

A third aspect of one or more embodiments of the present invention is a method for controlling a plant disease. The method for controlling a plant disease of one or more embodiments of the present invention is characterized in that a plant disease of a target plant is controlled by applying the plant disease control composition according to the second aspect to the target plant.

According to the method for controlling a plant disease of one or more embodiments of the present invention, plant diseases caused by bacteria, particularly bacteria of the genus Xanthomonas, in a target plant can be controlled.

### 3-2. Method

The method for controlling a plant disease of one or more embodiments of the present invention comprises a contact step as an essential step.

The “contact step” is a step of bringing the plant disease control composition according to the second aspect into contact with a target plant. This step is basically in accordance with “2-3. Application method” for the plant disease control composition of the second aspect.

In one or more embodiments, the term “contact” means that the plant disease control composition is brought into contact with a target plant. More specifically, it means that the bacteriolytic agent according to the first aspect, which is an active ingredient of the plant disease control composition, i.e., a phage, is brought into contact with a plant body of a target plant, particularly a site infected with target bacteria or a site at risk of infection. The purpose of this step is to allow the phage, which is an active ingredient, to infect the target bacterium, whereby the target bacteria are lysed. As a result, an effect of controlling a plant disease caused by the target bacteria can be exhibited.

The contact may be either direct contact or indirect contact. In one or more embodiments, the direct contact means that the plant disease control composition is brought into direct contact with a predetermined site of a target plant. Specifically, for example, the direct contact means that the plant disease control composition in a liquid or gel form is applied to, sprayed to, scattered to, or used to immerse a plant body of a target plant. In this case, the plant is brought into contact mainly at a site such as a leaf, a flower, a fruit, a stem, a branch, and/or a trunk. On the other hand, in one or more embodiments, the indirect contact means that the plant disease control composition is brought into contact with a predetermined site of a target plant via a medium. For example, the indirect contact means that the plant disease control composition in a granular form is applied to the soil around the root of a target plant. The phage, which is an active ingredient, is transported through water or the like in the soil, and is absorbed through the root.

### 3-3. Effect

The phage, which is an active ingredient of the composition obtained by one or more embodiments of the present invention, can efficiently kill target bacteria, and thus is useful for preventing or suppressing diseases caused by the target bacteria. Disease can also be diagnosed by detecting and identifying the target bacterium based on its specific bacteriolytic activity. Examples of drugs conventionally used for bacteria of the genus Xanthomonas include copper agents and antibiotics, but these drugs may cause phytotoxicity or disruption of the bacterial flora balance. For example, several strains of Pseudomonas fluorescens have been demonstrated to have plant growth-promoting effects (Haas D., Defago G., Nature Reviews in Microbiology, 2005, 3 (4), 307-19), and it is highly likely that copper-based agents or antibiotic-based agents will also kill such bacteria symbiotic with plants. On the other hand, since the phage is an organism-derived substance, no manifestation of phytotoxicity has been reported, and since the specificity is high, the influence on the bacterial flora balance is extremely limited.

### 4. Method for Identifying Bacteria of Genus Xanthomonas

### 4-1. Overview

A fourth aspect of one or more embodiments of the present invention is a method for identifying bacteria of the genus Xanthomonas. The identification method of one or more embodiments of the present invention is characterized in that a bacterium of the genus Xanthomonas is identified by utilizing the host specificity of the phage constituting the bacteriolytic agent according to the first aspect.

According to one or more embodiments of the present invention, it is possible to determine and identify whether or not unidentified plant pathogenic bacteria causing a plant disease are bacteria of the genus Xanthomonas.

### 4-2. Method

The identification method of one or more embodiments of the present invention comprises a culturing step, a mixing step, a mixture-culturing step, and a determination step as essential steps, and further includes an isolation step as an optional step. Hereinafter, each step will be described.

### (1) Isolation Step

The “isolation step” is a step of isolating a test bacterium from a plant tissue affected by a plant disease. This step is an optional step and may be performed as necessary.

The term “test bacterium” refers to a plant pathogenic bacterium to be subjected to the method for identifying bacteria of the genus Xanthomonas according to the fourth aspect of one or more embodiments of the present invention, the species of which has not been identified.

The plant tissue may be any site of a plant where a plant disease has developed, but is particularly a site where symptoms of the plant disease are significantly observed. For example, in the case of a peach that has developed peach bacterial spot, a leaf showing the disease may be used.

In order to isolate test bacteria from a collected plant tissue, a specimen in which a disease is found may be immersed in a solvent such as water and extracted, and the specimen may be fragmented or crushed at the time of extraction. Subsequently, the extract may be streaked on an agar culture medium, and a single colony may be picked.

### (2) Culturing Step

The “culturing step” is a step of culturing the isolated test bacteria to obtain a culture. The test bacterium may be cultured by a method known in the art.

The “culture” is obtained by culturing a test bacterium, and may be either liquid or solid.

In this step, since the test bacteria are in an unidentified state, it is desirable to use a culture medium capable of widely culturing plant pathogenic bacteria as the culture medium used in this step. A culture medium capable of culturing at least bacteria of the genus Xanthomonas, which are identification target bacteria of one or more embodiments of the present invention, is used. Such a culture medium may be, for example, a culture medium containing one or more components selected from protein enzymatic decomposition products such as peptone and tryptone, biological extracts such as potato dextrose and yeast extract, amino acids such as glutamic acid or salts thereof, sugars such as glucose and sucrose, and inorganic salts such as sodium chloride, magnesium chloride, and potassium dihydrogen phosphate. Specific culture media and compositions include LB culture medium (tryptone, yeast extract, NaCl), YPG culture medium (yeast extract, peptone, glucose), PD culture medium (potato dextrose), Suwa culture medium supplemented with peptone (sucrose, glutamate, peptone) and the like.

The isolated test bacteria are inoculated into the culture medium and cultured under appropriate culture conditions. The culture can be obtained by culturing with stirring under a culture condition of, for example, 20 to 40° C., 20 to 30° C., 22 to 28° C., or 24 to 26° C. The culture time is not limited, and for example, the culture may be performed until the turbidity at a wavelength of 600 nm reaches about 1.0. By this step, a culture liquid of the test bacteria is obtained. The culture may be a multi-stage culture of two or more stages. For example, further culturing may be performed after a soft agar-containing liquid culture medium is added to a culture liquid obtained after culturing in a liquid culture medium, and the mixture is poured onto a solid culture medium such as an agar culture medium and solidified.

### (3) Mixing Step

The “mixing step” is a step of mixing the culture obtained in the culturing step with the bacteriolytic agent according to the first aspect to obtain a mixture.

The “mixture” is a mixture of the culture and the bacteriolytic agent, and may be a liquid or a solid.

The mixing method is not particularly limited as long as the culture and the bacteriolytic agent can be mixed. The bacteriolytic agent according to the first aspect may be administered in a solid state or in a liquid state in which it is suspended in water or a liquid culture medium.

When both the culture and the bacteriolytic agent are liquid, the volumetric ratio of culture to bacteriolytic agent may be 1:9, 2:8, 3:7, 4:6, 5:5, 6:4, 7:3, 8:2, or 9:1. After the administration, the culture and the bacteriolytic agent may be sufficiently mixed by stirring or the like. On the other hand, when a soft agar-containing liquid culture medium is laminated as described above, the culture is solid. In this case, the mixture may be obtained by dropping the bacteriolytic agent onto the solid culture medium like a gel surface to mix them on the solid culture medium.

### (4) Mixture-Culturing Step

The “mixture-culturing step” is a step of culturing the mixture under predetermined conditions.

When the mixture is cultured, a soft agar-containing liquid culture medium may be added to the mixture, and the mixture may be poured onto a solid culture medium such as an agar culture medium and solidified, followed by further culturing.

The basic procedure of this step is the same as that of the culturing step. In this step, it is desirable to perform culture based on the so-called plaque assay method so that the presence or absence of lysis of the test bacteria by the phage constituting the bacteriolytic agent can be easily confirmed in the determination step described below, although the method is not limited thereto. For example, a part of the liquid mixture may be mixed with a soft agar culture medium having the same composition, and then poured onto an agar culture medium having the same composition before the soft agar culture medium is solidified, and spread over the entire culture medium. Thereafter, the mixture may be cultured under the same conditions as in the culturing step.

### (5) Determination Step

The “determination step” is a step of determining that the test bacteria are bacteria of the genus Xanthomonas when the test bacteria after the culturing step are lysed.

The determination of the presence or absence of lysis is not limited. For example, when the determination is based on the plaque assay method, the determination may be made based on the presence or absence of plaque formation. When plaques are present in the soft agar culture medium which has been spread and solidified on the agar culture medium after the above-mentioned mixture-culturing step, it indicates that the test bacteria have been lysed by infection with the phage constituting the bacteriolytic agent of one or more embodiments of the present invention. Therefore, the test bacteria can be determined to be bacteria of the genus Xanthomonas. On the other hand, when the test bacteria proliferate on the entire agar culture medium and no plaques are present, it can be determined that the test bacteria are not bacteria of the genus Xanthomonas.

In order to perform more accurate determination, a negative control to be mixed with a culture medium not containing the bacteriolytic agent in the mixture-culturing step and/or a positive control using identified bacteria of the genus Xanthomonas in the culturing step instead of the test bacteria may be simultaneously prepared to confirm that no plaques are formed in the negative control and plaques are observed in the positive control.

### 4-3. Effect

According to the method for identifying bacteria of the genus Xanthomonas of one or more embodiments of the present invention, it is possible to identify whether or not a plant disease is caused by bacteria of the genus Xanthomonas.

Further, according to the method for identifying bacteria of the genus Xanthomonas of one or more embodiments of the present invention, it is possible to detect whether or not bacteria of the genus Xanthomonas are present in a lesion of a plant which has developed a plant disease which is presumed to be caused by bacteria of the genus Xanthomonas.

### EXAMPLES

### Example 1: Isolation of Novel Bacteriophages and Bacteriolytic Activity Thereof (1)

### (Objective)

To isolate a new bacteriophage having bacteriolytic activity against pathogenic bacteria of plant diseases and to verify the bacteriolytic activity against the pathogenic bacteria of the plant diseases.

(Methods and results)

### (1) Obtaining and Culturing Plant Pathogenic Bacteria

In this example, all of the target plant pathogenic bacteria were obtained from the National Agriculture and Food Research Organization (NARO). Each strain used in this Example is described in Table 2 together with the accession numbering of the National Agriculture and Food Research Organization.

TABLE 2

| | Species | Isolation | Collection |
|---|---|---|---|
| ID | name | source | location |
| MAFF No. | Xanthomonas arboricola | Peach | Yamanashi |
| 211971 | pv. pruni | | |
| MAFF No. | Xanthomonas arboricola | Peach | Fukushima |
| 311351 | pv. pruni | | |
| MAFF No. | Xanthomonas arboricola | Walnut | Nagano |
| 212146 | pv. juglandis | | |
| MAFF No. | Xanthomonas campestris | Tomato | Tokushima |
| 301256 | pv. vesicatoria | | |
| MAFF No. | Xanthomonas campestris | Lettuce | Wakayama |
| 301352 | pv. vitians | | |
| MAFF No. | Xanthomonas arboricola | Peach | Yamanashi |
| 311622 | pv. pruni | | |
| MAFF No. | Xanthomonas citri | Mandarin | Wakayama |
| 301078 | subsp. citri | | |
| NCIMB-ID | Pseudomonas fluorescens | Chicken egg | US |
| 10460 | | | |

As a control, an environmentally beneficial Pseudomonas fluorescens strain reported to have plant growth promoting effects was obtained from the NCIMB Institute in the UK National Culture Collection (UKNCC) (NCIMB-ID: 10460).

A liquid culture medium (YPG Broth) in which peptone (1 g), yeast extract (1 g), and glucose (2 g) were dissolved in H₂O (1 L) and autoclaved was used for culturing the bacteria of the genus Xanthomonas and the Pseudomonas fluorescens sp. In addition, as an agar culture medium, an agar culture medium obtained by adding 15 g agar per liter of the above Broth (YPG Broth) and autoclaving the mixture was used (referred to as “YPG Agar” when YPG Broth was used). Further, as a soft agar culture medium (Top Agar) to be laminated as an upper layer on the agar culture medium, Top Agar prepared by adding 5 g of agarose per liter of the above-mentioned Broth followed by autoclaving was stored at about 50° C. and used as required.

Each of the above-mentioned strains delivered in a dry powder state was suspended in Broth (0.1 mL), and then streaked and cultured on Agar (YPG Agar) at 25° C. to isolate a single colony. The isolated colony was inoculated into Broth and cultured with shaking at 25° C., and this was used as a preculture liquid. In the main culture, the preculture liquid was inoculated into Broth and cultured at 25° C. for 10 hours to 30 hours until the turbidity (optical density at 600 nm) reached about 1.0. The culture liquid after culturing was used as a bacterial liquid as it was.

### (2) Isolation and Purification of First Phage

The novel phage was isolated from natural sewage or soil obtained in Japan. The phage was isolated by a conventional plaque assay method. First, sewage from a pond, a lake, or the like, or sewage obtained by suspending soil in water was filtered through a 0.45 μm filter to prepare a phage-containing liquid. Subsequently, the bacterial liquid and the phage-containing liquid were mixed in equal amounts and allowed to stand at room temperature for about 10 minutes. The bacteria/phage liquid mixture (0.2 mL) was then added to Top Agar (3 mL), briefly mixed on a vortex mixer, and then poured onto Agar. After Top Agar was solidified, static culture was performed at 25° C. for about 12 hours. A lytic plaque was allowed to form on a lawn of the bacteria formed by the culture. Thereafter, the gel of the plaque portion was aspirated using a truncated tip, and a phage having bacteriolytic activity against the bacteria of the genus Xanthomonas was isolated. Then, the phage-containing liquid containing the isolated phage at a high concentration was used instead of the sewage, and this procedure was repeated to purify the phage.

A total of three kinds of novel phages were isolated from natural sewage and soil by a technique for detecting a lysis plaque formed on a soft agar culture medium in which any of the above-mentioned strains was amplified. One of the novel phages isolated in Example 1 is referred to as a “first phage”.

The isolated phage was suspended in SM Buffer and passed through a 0.2 μm filter to collect a phage-containing liquid. This phage-containing liquid was mixed with the above-mentioned bacterial liquid under the above-mentioned conditions, and the phage was isolated again. This procedure was repeated several times to further purify the phage. The composition of SM Buffer is shown in Table 3.

TABLE 3

| | | |
|---|---|---|
| SM Buffer | Final | Add to 1 L |

| | | | | |
|---|---|---|---|---|
| NaCl | 0.1 | M | 5.8 | g |
| Mg₂SO₄•7H₂O | 10 | mM | 1 | g |
| 1M Tris-HCl | 50 | mM | 50 | mL |
| pH 8.0 | | | | |

| | | | |
|---|---|---|---|
| Gelatin | 0.1% | 0.1 | g |

### (3) Amplification and Purification of First Phage

In order to amplify and purify the isolated and purified first phage, a plate lysate (PL) method which is an amplification method using a plaque assay method was performed. For formation of many plaques on Agar, the bacteria/phage liquid mixture was adjusted, mixed with Top Agar, and then spread and cultured on YPG Agar. Thereafter, SM Buffer (3 mL) was added onto Top Agar on which plaques were formed, and the mixture was shaken at 25° C. for about 30 minutes, and the supernatant was passed through a 0.2 μm filter to recover a recovery liquid containing the phage.

Purification of the first phage was carried out by adding PEG 6000 (1 g, final concentration: 10%) and NaCl (0.4 g, final concentration: 4%) to the recovered solution (10 mL) to dissolve the phage, and rotating the solution overnight at 4° C. using a rotator. Thereafter, the mixture was centrifuged at ×15,000 g at 4° C. for 60 minutes to remove the supernatant. The recovered pellet was resuspended in SM Buffer (0.5 mL). Subsequently, chloroform (0.5 mL) was added, and the mixture was vigorously stirred and allowed to stand on ice for 6 hours. After centrifugation at ×8,000 g at 4° C. for 10 minutes, the upper layer was carefully collected to obtain a purified phage liquid. The concentration of a purified phage liquid is generally represented by a titer [PFU/mL] based on the number of plaques (Plaque Forming Unit, PFU) by a plaque assay method, and serves as an indicator of bacteriolytic activity. The titer of the prepared purified first phage liquid was determined by a plaque assay method using appropriately diluted solutions, and a titer of 108 PFU/mL or higher was confirmed.

### (4) Host Range Evaluation of First Phage

The host range of the first phage was evaluated by a spot test method. The bacterial liquid (0.1 mL) alone was added to and mixed with Top Agar (3 mL), and the mixture was then poured onto Agar, spread over the entire plate, and solidified. As the bacterial liquid, in addition to the bacterial liquid of each bacterium of the genus Xanthomonas shown in Table 2 prepared in the above (1), a bacterial liquid of Pseudomonas fluorescens for control was also prepared. Then, about 5 μL of the purified phage liquid was dropped, followed by static culture at 25° C. for about 12 hours. When a portion on the plate on which the bacterial lawn was formed and at a place where the phage was dropped turned clear in the form of a circle (about 1 cm in diameter), it was determined that the dropped phage had bacteriolytic activity against the strain.

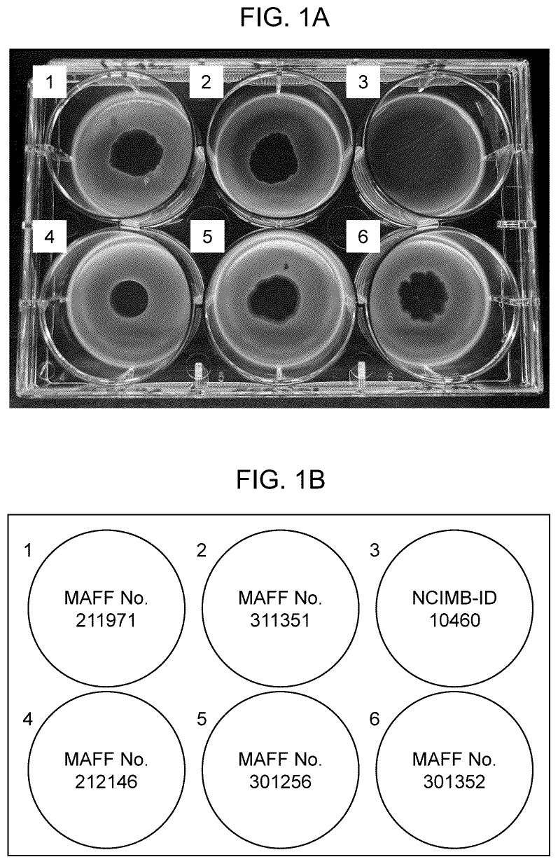

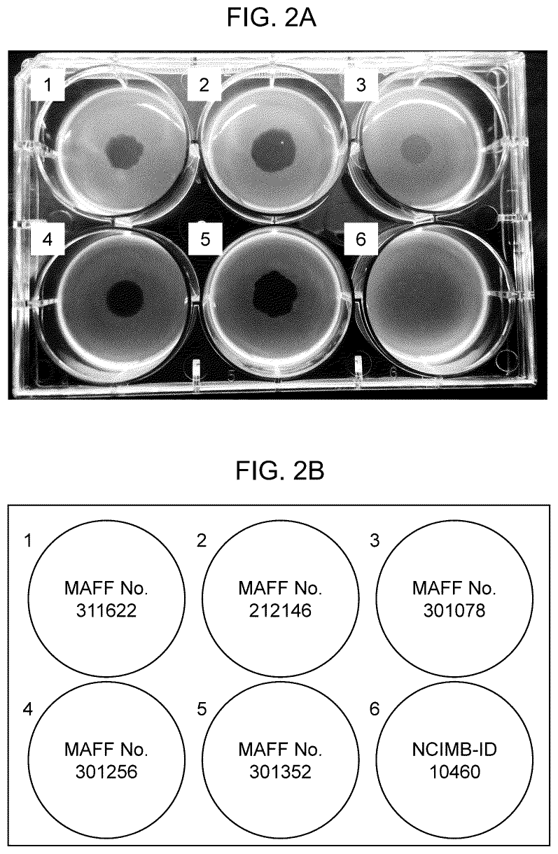

An example of the results is shown in FIGS. 1 and 2. When the first phage exhibits bacteriolytic activity, the bacterial lawn formed on the plate turns clear only in an area to which the purified phage liquid has been dropped. One type of the first phage obtained by one or more embodiments of the present invention exhibited bacteriolytic activity against all of the strains belonging to the three species of Xanthomonas arboricola (including pv. pruni and pv. juglandis), Xanthomonas campestris (including pv. vesicatoria and pv. vitians), and Xanthomonas citri shown in Table 2. On the other hand, it was also confirmed that it did not exhibit bacteriolytic activity against Pseudomonas fluorescens. There are reports of phages showing activity against two or more species of the genus Xanthomonas, but the above-mentioned pattern has not been reported (Nakayinga R. et al., BMC Microbiology, 2021, 21:291). This suggests that the first phage isolated exhibits broad bacteriolytic activity against bacteria of the genus Xanthomonas.

This is a result showing the possibility that the first phage can be applied to various plant diseases. For example, the first phage is suggested to be useful for controlling peach bacterial spot, walnut bacterial blight, tomato bacterial blight, lettuce bacterial blight, mandarin orange bacterial canker and the like, which are caused by bacteria of the genus Xanthomonas, and the first phage is also expected to have a high value in industrial utilization.

### (5) Genomic Analysis of First Phage

The genomic DNA sequence was determined and analyzed for the first phage.

### (i) Preparation and Sequencing of Genomic DNA of First Phage

The genome of the first phage was extracted using a TURBO DNA-Free™ kit (Thermo Fisher Scientific Inc.). The genomic DNA derived from the host bacterium as a contaminant was removed by the treatment according to the manual attached to the kit. Thereafter, phage coat molecules were degraded by the proteinase K treatment using NucleoSpin® Virus (Machery-Nagel) in accordance with the attached manual. A genomic DNA solution of the first phage was prepared through genomic DNA purification using a silica spin column. Thereafter, the concentration of the genomic DNA was measured using Qubit dsDNA HS Assay kit (Thermo Fisher Scientific Inc.), and 50 μL of genomic DNA solutions were prepared so as to have a final concentration of 0.2 ng/μL. Next, using Nextera XT DNA Library Prep (Illumina), fragmentation of the genome of the first phage and addition of adaptor sequences by PCR were performed by a treatment according to the attached manual. Next, electrophoresis was performed on Bioanalyzer (Agilent Technologies) using Agilent High Sensitivity DNA Kit (Agilent Technologies), and the average bp size of the sample was measured to determine the concentration of DNA fragments. Finally, a measurement sample was prepared by a treatment using a Miseq Reagent kit (Illumina) according to the attached manual, and measurement was performed using a next-generation sequencer Miseq (Illumina). The obtained data were subjected to preprocessing (trimming and the like) using CLC genomics workbench (Qiagen). Then, de novo assembly was performed to obtain a contig sequence corresponding to the genome sequence of the phage.

### (ii) Bioinformatics Analysis Based on Genomic Sequence Information

Based on the base sequence (SEQ ID NO: 1) of the genomic DNA of the first phage obtained in (i) above, a search for similar DNA sequences and confirmation of sequence identity were performed using the BLAST server (https://blast.ncbi.nlm.nih.gov/Blast.cgi) provided by NCBI. When a range of 90% or more of the entire length (Query Cover) was searched for known sequences having a high sequence identity, the sequence having the highest numerical value of sequence identity was the genomic DNA sequence of Pseudomonas phage vB_Pae_TR (accession code: OL802211.1), and had a sequence identity of 99.07% over a range of 93% of the entire length of SEQ ID NO: 1.

The sequence identity is a numerical value for a range automatically aligned by the analysis server with respect to the entire length of the genome of the first phage. The numerical value in the range is indicated as Query Cover, and the sequence identity of 99.07% of vB_Pae_TR is a numerical value calculated by limiting to a region of 93% of the entire length of ΦX-33. Thus, a sequence identity over the entire length is difficult to calculate, but is at least estimated to be less than 99.07%, and usually equal to or less than 93%.

It was revealed that the target bacterium of the phage vB_Pae_TR having the genome of the known sequence is a bacterium of the genus Pseudomonas. Therefore, it can be said that it was difficult to identify the target bacterium of the first phage isolated in this Example based on the sequence identity with the genome sequence of a known phage. At the same time, it is considered that a phage having a higher sequence identity to the full length than vB_Pae_TR (a phage showing a higher sequence identity over a wider range) is highly likely to be a phage having a host range similar to that of the first phage isolated in the present Example, that is, a phage using various bacteria of the genus Xanthomonas as host bacteria.

From the above results, it was suggested that the first phage is a completely novel phage having a host range of the genus Xanthomonas which has not been reported so far.

### Example 2: Isolation of Novel Bacteriophage and Bacteriolytic Activity Thereof (2)

### (Objective)

To isolate a new bacteriophage having bacteriolytic activity against pathogenic bacteria of plant diseases and to verify the bacteriolytic activity against the pathogenic bacteria of the plant diseases.

### (Methods and Results)

### (1) Obtaining and Culturing Plant Pathogenic Bacteria

In this Example, plant pathogenic bacteria as target bacteria were bacteria of the genus Xanthomonas, and all the strains were obtained from the National Agriculture and Food Research Organization (NARO). The strains used in Examples 7 and 8 are described in Table 4 together with the accession numbering (MAFF Nos.) of the National Agriculture and Food Research Organization.

TABLE 4

| | Species | Isolation | Collection |
|---|---|---|---|
| ID | name | source | location |
| MAFF No. | Xanthomonas arboricola | Peach | Yamanashi |
| 311622 | pv. pruni | | |
| MAFF No. | Xanthomonas arboricola | Walnut | Nagano |
| 212146 | pv. juglandis | | |
| MAFF No. | Xanthomonas campestris | Lettuce | Wakayama |
| 301352 | pv. vitians | | |
| MAFF No. | Xanthomonas campestris | Fescue | Ibaraki |
| 106642 | pv. campestris | | |
| MAFF No. | Xanthomonas campestris | Tomato | Iwate |
| 730097 | pv. vesicatoria | | |
| MAFF No. | Xanthomonas arboricola | Walnut | Nagano |
| 212147 | pv. juglandis | | |
| MAFF No. | Xanthomonas arboricola | Walnut | Nagano |
| 212148 | pv. juglandis | | |
| MAFF No. | Xanthomonas arboricola | Walnut | Nagano |
| 212149 | pv. juglandis | | |
| MAFF No. | Xanthomonas campestris | Tomato | Tokushima |
| 301256 | pv. vesicatoria | | |
| MAFF No. | Xanthomonas campestris | Tomato | Shizuoka |
| 301294 | pv. vesicatoria | | |
| MAFF No. | Xanthomonas campestris | Tomato | Tokushima |
| 301257 | pv. vesicatoria | | |
| MAFF No. | Xanthomonas campestris | Bell pepper | Tokushima |
| 301260 | pv. vesicatoria | | |
| MAFF No. | Xanthomonas campestris | Lettuce | Nagano |
| 301356 | pv. vitians | | |
| MAFF No. | Xanthomonas campestris | Lettuce | Chiba |
| 301354 | pv. vitians | | |
| MAFF No. | Xanthomonas campestris | Lettuce | Fukuoka |
| 301358 | pv. vitians | | |
| NCIMB-ID | Pseudomonas fluorescens | Chicken egg | US |
| 10460 | | | |

As a control, a strain of Pseudomonas fluorescens, which is a bacterium of the genus Pseudomonas, was obtained from the Institute NCIMB in the UK National Culture Collection (UKNCC) (NCIMB-ID: 10460).

The bacteria of the genus Xanthomonas and Pseudomonas fluorescens were cultured according to the method described in “(1) Obtaining and culturing plant pathogenic bacteria” in Example 1.

### (2) Isolation of the Second Phage and Purification Thereof

The novel phage was isolated from natural sewage or soil obtained in Japan. The isolation method and the purification method were in accordance with the methods described in “(2) Isolation and purification of first phage” in Example 1. As a result, one novel phage was isolated from natural sewage or soil. The novel phage isolated in Example 2 is referred to as a “second phage”.

The isolated phage was suspended in SM Buffer and passed through a 0.2 μm filter to collect a phage-containing liquid. This phage-containing liquid was mixed with the above-mentioned bacterial liquid under the above-mentioned conditions, and the phage was isolated again. This procedure was repeated several times to purify the phage.

### (3) Amplification and Purification of Second Phage

In order to amplify and purify the isolated and purified second phage, a plate lysate (PL) method was performed. The specific method was in accordance with the method described in “(3) Amplification and purification of phage” in Example 1. The titer of the prepared purified second phage liquid was determined by a plaque assay method using appropriately diluted solutions, and was confirmed to be 108 PFU/mL or higher.

### (4) Host Range Evaluation of Second Phage

The host range of the second phage was evaluated by a spot test method. The basic operation was in accordance with the method described in “(4) Evaluation of host range of first phage” in Example 1.

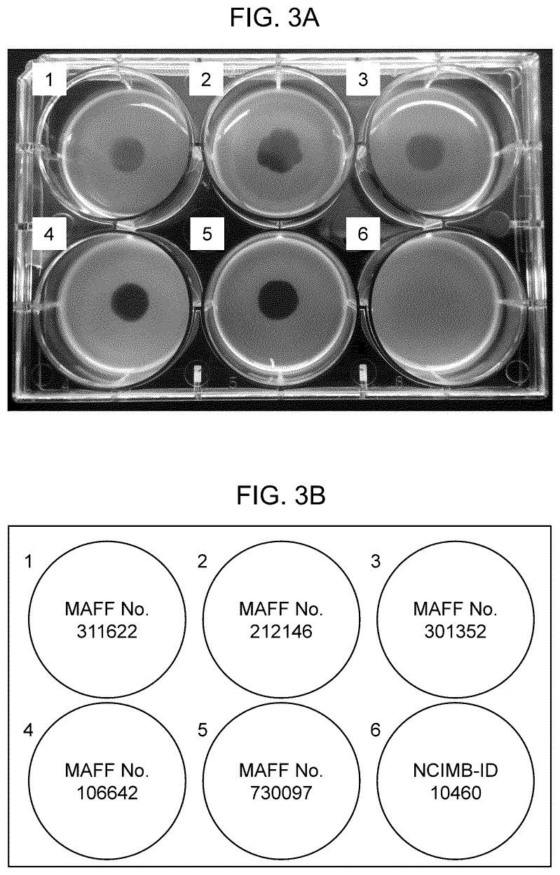

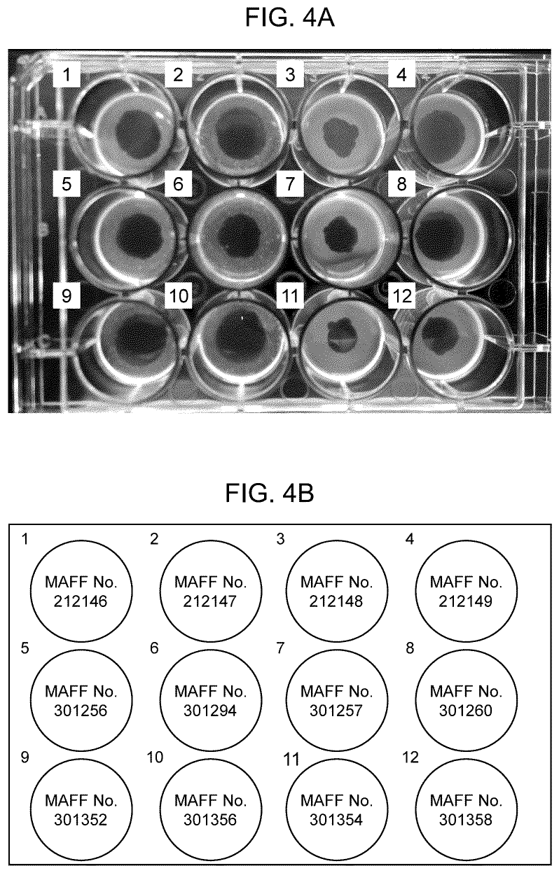

An example of the results is shown in FIGS. 3 and 4. When the second phage exhibits bacteriolytic activity, the bacterial lawn formed on the plate turns clear only in an area to which the purified phage liquid has been dropped. The second phage obtained by one or more embodiments of the present invention exhibited bacteriolytic activity against all of the bacteria of the genus Xanthomonas shown in Table 4. On the other hand, the second phage did not exhibit bacteriolytic activity against Pseudomonas fluorescens. A phage exhibiting a broad bacteriolytic activity against all bacterial species and pathovars as shown in Table 4 has not been known so far. This suggests that the isolated second phage exhibits a broad bacteriolytic activity against bacteria of the genus Xanthomonas.

Similarly to the first phage, the second phage is also highly likely to be applicable to various plant diseases. This result suggests that the second phage is useful for controlling, for example, peach bacterial spot, walnut bacterial blight, tomato bacterial blight, lettuce bacterial blight, and the like caused by bacteria of the genus Xanthomonas.

### (5) Genomic Analysis of Second Phage

The genomic DNA sequence was determined and analyzed for the second phage.

### (i) Preparation and Sequencing of Genomic DNA of Second Phage

The genome of the second phage was extracted using a TURBO DNA-Free™ kit (Thermo Fisher Scientific). The basic operation was in accordance with the method described in “(5) Genomic analysis of first phage, (i) Preparation and sequencing of genomic DNA of first phage” in Example 1.

### (ii) Bioinformatics Analysis Based on Genomic Sequence Information

Based on the base sequence (SEQ ID NO: 2) of the genomic DNA of the second phage obtained in (i) above, a search for similar DNA sequences and confirmation of sequence identity were performed using the BLAST server provided by NCBI. As a result of the search, a partial sequence of the genomic DNA of a phage targeting a bacterium of the genus Xanthomonas (accession code: MT664984.1), which is called Xp12, had the highest similarity score, and had a sequence identity of 99.03% over a range of 99% of the entire length of SEQ ID NO: 2. From this result, the sequence identity to the full length of SEQ ID NO: 2 is estimated to correspond to a numerical value of about 98.04%. However, only Xanthomonas oryzae is known as a bacterial species against which the phage Xp12 exhibits bacteriolytic activity (Nakayinga R. et al., BMC Microbiology, 2021, 21:291).

The total length of the genomic DNA sequence of Xp12 is 63,783 bases, whereas the total length of the genomic DNA sequence of the second phage isolated in this Example was 35,321 bases, which was about a half of the total length of the genomic DNA sequence of Xp12. As a result, contrary to the above, when the sequence identity based on the genomic DNA sequence of Xp12 was calculated using GENETYX-NGS included in the genetic information processing software GENETYX (https://www.genetyx.co.jp/), the base sequence (SEQ ID NO: 2) of the genomic DNA of the second phage had a sequence identity of 98.34% over a range of 54% of the entire length of the genomic DNA sequence of Xp12. From this result, it was estimated that the sequence identity between the full-length genomic DNA sequences of both phages was about 53.10%. Therefore, the genomic DNA sequence of the second phage obtained in this Example has only a sequence homologous to a part of the genomic DNA sequence of Xp12, and it cannot be said that the degree of similarity is high as a whole.

These differences in the genomic DNA sequences were further analyzed by comparing the genomic DNA sequences of Xp 12 and the phage of one or more embodiments of the present invention using MUMmer included in genetic information processing software GENETYX (https://www.genetyx.co.jp/). As a result, a sequence homologous to the region from 12,894 to 41,320 of the genomic DNA sequence of Xp12 was not detected in the base sequence of SEQ ID NO: 2, and it was found that the region was deleted in the genomic DNA sequence of the second phage. The region contained a gene presumed to be a tail fiber gene based on the sequence identity (accession code: QNN97189.1), a tail fiber gene (accession code: QNN97196.1), and an endolysin gene (accession code: QNN97201.1). These genes are deeply involved in the process of infection of a host. Also from this point of view, it was suggested that the second phage was largely different from Xp12 in form and properties, and this difference led to the difference in host range. In addition, there were phages having a base sequence with a relatively high similarity score to the base sequence of SEQ ID NO: 2, but all of them had a full length of more than 60,000 bases as in the case of the genomic DNA sequence of Xp12, and were phages having a high similarity score to the full length of the genomic DNA sequence of Xp12.

The search range was further expanded to search for sequences having a sequence identity of 80% or more over a range of 80% or more of the entire length (Query Cover) with respect to the genomic DNA sequence of the second phage. However, a known sequence having a high similarity score with respect to the genomic DNA sequence of the second phage and having a total genomic DNA sequence length of 60,000 bases or less was not detected on the BLAST server provided by NCBI.

From the above, it was suggested that the second phage was a completely novel phage whose properties were changed by deletion of a part of its genomic DNA.

### Example 3: Isolation of Novel Bacteriophage and Bacteriolytic Activity Thereof (3)

### (Objective)

To isolate a new bacteriophage having bacteriolytic activity against pathogenic bacteria of plant diseases and to verify the bacteriolytic activity against the pathogenic bacteria of the plant diseases.

### (Methods and Results)

### (1) Obtaining and Culturing Plant Pathogenic Bacteria

In this example, the plant pathogenic bacteria were bacteria of the genus Xanthomonas, and all strains were obtained from the National Agriculture and Food Research Organization (NARO). Each strain used in this Example is described in Table 5 together with the accession numbering (MAFF No.) of the National Agriculture and Food Research Organization.

TABLE 5

| | Species | Isolation | Collection |
|---|---|---|---|
| ID | name | source | location |
| MAFF No. | Xanthomonas arboricola | Walnut | Nagano |
| 212146 | pv. juglandis | | |
| MAFF No. | Xanthomonas arboricola | Walnut | Nagano |
| 212147 | pv. juglandis | | |
| MAFF No. | Xanthomonas campestris | Tomato | Shizuoka |
| 301294 | pv. vesicatoria | | |
| MAFF No. | Xanthomonas campestris | Tomato | Tokushima |
| 301257 | pv. vesicatoria | | |
| MAFF No. | Xanthomonas campestris | Cabbage | Ibaraki |
| 106692 | pv. campestris | | |
| MAFF No. | Xanthomonas campestris | Broccoli | Ibaraki |
| 106765 | pv. campestris | | |
| MAFF No. | Xanthomonas campestris | Lettuce | Gunma |
| 301359 | pv. vitians | | |
| NCIMB-ID | Pseudomonas fluorescens | Chicken egg | US |
| 10460 | | | |

As a control, a strain of Pseudomonas fluorescens, which is a bacterium of the genus Pseudomonas, was obtained from the Institute NCIMB in the UK National Culture Collection (UKNCC) (NCIMB-ID: 10460).

The bacteria of the genus Xanthomonas and Pseudomonas fluorescens were cultured according to the method described in “(1) Obtaining and culturing plant pathogenic bacteria” in Example 1.

### (2) Isolation of Third Phage and Purification Thereof

The novel phage was isolated from natural sewage or soil obtained in Japan. The isolation method and the purification method were in accordance with the methods described in “(2) Isolation and purification of first phage” in Example 1. As a result, one novel phage was isolated from natural sewage or soil. The novel phage isolated in Example 3 is referred to as a “third phage”.

The isolated phage was suspended in SM Buffer and passed through a 0.2 μm filter to collect a phage-containing liquid. This phage-containing liquid was mixed with the above-mentioned bacterial liquid under the above-mentioned conditions, and the phage was isolated again. This procedure was repeated several times to purify the phage.

### (3) Amplification and Purification of Third Phage

In order to amplify and purify the isolated and purified third phage, a plate lysate (PL) method was performed. The specific method was in accordance with the method described in “(3) Amplification and purification of phage” in Example 1. The titer of the prepared purified third phage liquid was determined by a plaque assay method using appropriately diluted solutions, and was confirmed to be 108 PFU/mL or higher.

### (4) Host Range Evaluation of Third Phage

The host range of the third phage was evaluated by a spot test method. The basic operation was in accordance with the method described in “(4) Evaluation of host range of first phage” in Example 1.

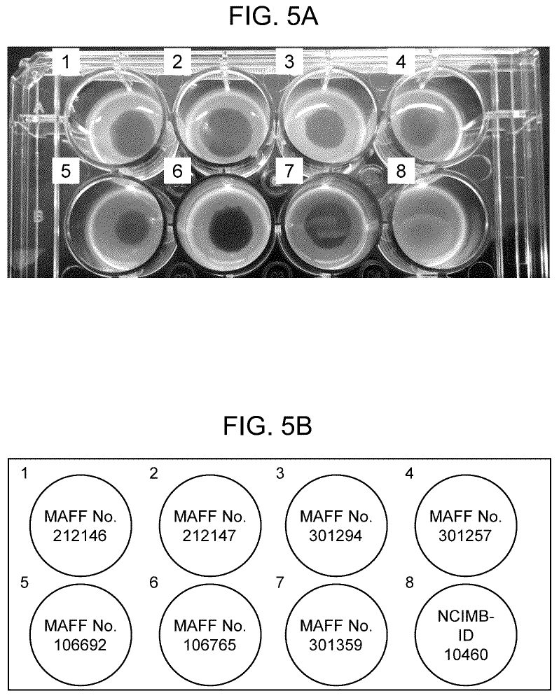

An example of the results is shown in FIG. 5. When the third phage exhibits bacteriolytic activity, the bacterial lawn formed on the plate turns clear only in an area to which the purified phage liquid has been dropped. The third phage obtained by one or more embodiments of the present invention exhibited bacteriolytic activity against all of the bacteria of the genus Xanthomonas shown in Table 5. On the other hand, the third phage did not exhibit bacteriolytic activity against Pseudomonas fluorescens. A phage which exhibits broad bacteriolytic activity against all bacterial species and pathovars as shown in Table 5 has not been known so far. This suggests that the isolated third phage exhibits broad bacteriolytic activity against bacteria of the genus Xanthomonas.

In addition, this result suggests that the third phage is useful for controlling walnut bacterial blight, tomato bacterial blight, lettuce bacterial blight, broccoli black rot, and the like, which are caused by bacteria of the genus Xanthomonas.

### (5) Genomic Analysis of Third Phage

The genomic DNA sequence was determined and analyzed for the third phage.

### (i) Preparation and Sequencing of Genomic DNA of Third Phage

The genome of the third phage was extracted using a TURBO DNA-Free™ kit (Thermo Fisher Scientific). The basic operation was in accordance with the method described in “(5) Genomic analysis of first phage, (i) Preparation and sequencing of genomic DNA of first phage” in Example 1.

### (ii) Bioinformatics Analysis Based on Genomic Sequence Information

Based on the base sequence (SEQ ID NO: 3) of the genomic DNA of the third phage obtained in (i) above, a search for similar DNA sequences and confirmation of sequence identity were performed using the BLAST server provided by NCBI. As a result of the search, the genomic DNA sequence of a phage (accession code: OK490494.1) targeting a bacterium of the genus Stenotrophomonas called vB_SmaS_P15 had the highest similarity score, and the sequence identity was 85.20% (Query Cover: 96.82% sequence identity over a range of 88% of the entire length of SEQ ID NO: 3). From this result, it is estimated that the sequence identity to the full length corresponds to a numerical value of about 85.20%. Similarly, a known phage having the second highest genomic DNA sequence identity (phage name: BUCT598, accession code: MW831865.1, sequence identity to the full length: about 85.18%) and known phages having the third highest genomic DNA sequence identity (phage names: BUCT703 and BUCT700, accession codes: OM735688.1 and OM735686.1, respectively, sequence identity to the full length: about 56.7% for both) each targeted bacteria of the genus Stenotrophomonas.

This result suggested that the third phage is a completely novel phage whose hosts are bacteria of the genus Xanthomonas.

### Example 4: Bacteriolytic Activity of Combination of Obtained Bacteriophages (1)

### (Objective)

To verify the effect of a novel bacteriophage, which has been shown to have bacteriolytic activity against pathogenic bacteria of plant diseases when used alone, on plant diseases when used in combination.

### (Method)

### (1) Spot Test Method

A spot test was performed in accordance with the method described in “(4) Evaluation of host range of first phage” in Example 1. In this example, strains of Xanthomonas cucurbitae (MAFF No. 673005) and Xanthomonas campestris pv. vitians (MAFF No. 301352), against which all the phages tested had been confirmed to exhibit bacteriolytic activity, were used.

As the purified phage liquids to be dropped, mixed solutions obtained by mixing equal amounts of the purified phage liquids prepared in Examples were used. Each mixed solution was prepared by appropriate dilution so that the total titer was equivalent to that in the case of single use. The phages used in this Example were as follows.

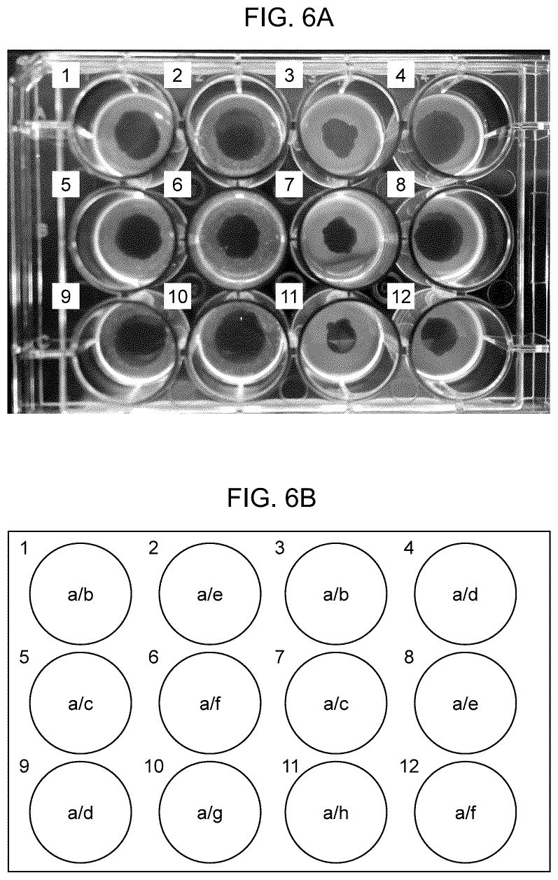

In this Example, the first phage having the genomic DNA sequence of SEQ ID NO: 1 (a in FIG. 6B) was used in combination with a phage having the genomic DNA sequence of SEQ ID NO: 4 (b in FIG. 6B), a phage having the genomic DNA sequence of SEQ ID NO: 5 (c in FIG. 6B), a phage having the genomic DNA sequence of SEQ ID NO: 6 (d in FIG. 6B), a phage having the genomic DNA sequence of SEQ ID NO: 7 (e in FIG. 6B), the second phage having the genomic DNA sequence of SEQ ID NO: 2 (f in FIG. 6B), the third phage having the genomic DNA sequence of SEQ ID NO: 3 (g in FIG. 6B), and a phage having the genomic DNA sequence of SEQ ID NO: 8 (h in FIG. 6B).

### (Results)

An example of the results is shown in FIG. 6. When a phage combination exhibits bacteriolytic activity, the bacterial lawn formed on the plate turns clear only in an area to which the purified phage liquid has been dropped. It was found that all the combinations of two phages tested exhibited at least as much bacteriolytic activity as each phage used alone (FIG. 6).

From the above results, it was confirmed that the phage of one or more embodiments of the present invention is useful when used alone or in combination.

### Example 5: Bacteriolytic Activity of Combination of Obtained Bacteriophages (2)

### (Objective)

To verify the effect of a novel bacteriophage, which has been shown to have bacteriolytic activity against pathogenic bacteria of plant diseases when used alone, on plant diseases when used in combination.

### (Method)

### (1) Spot Test Method

A spot test was performed in accordance with the method described in “(4) Evaluation of host range of first phage” in Example 1. In this example, strains of Xanthomonas cucurbitae (MAFF No. 673005) and Xanthomonas campestris pv. vitians (MAFF No. 301352), against which all the phages tested had been confirmed to exhibit bacteriolytic activity, were used.

As the purified phage liquids to be dropped, mixed solutions obtained by mixing equal amounts of the purified phage liquids prepared in Examples were used. Each mixed solution was prepared by appropriate dilution so that the total titer was equivalent to that in the case of single use. The phages used in this Example were as follows.

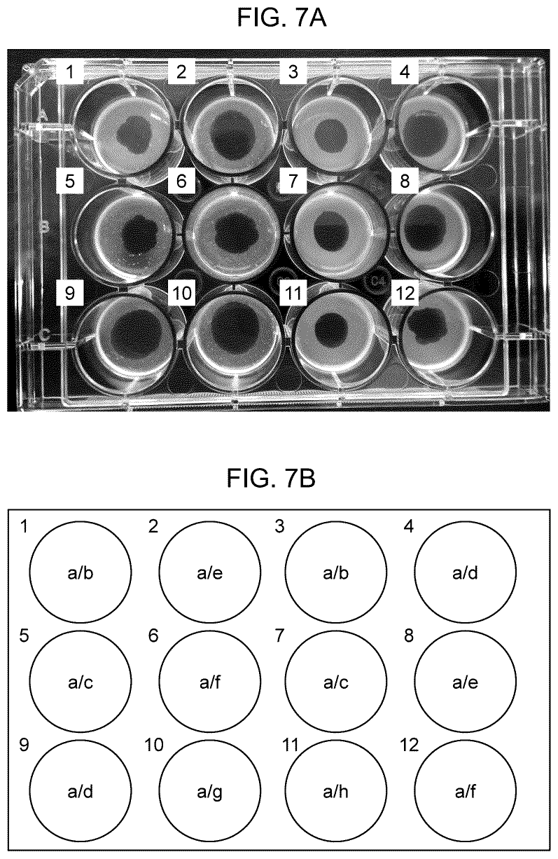

In this Example, the second phage having the genomic DNA sequence of SEQ ID NO: 2 (a in FIG. 7B) was used in combination with the phage having the genomic DNA sequence of SEQ ID NO: 4 (b in FIG. 7B), the phage having the genomic DNA sequence of SEQ ID NO: 5 (c in FIG. 7B), the phage having the genomic DNA sequence of SEQ ID NO: 6 (d in FIG. 7B), the phage having the genomic DNA sequence of SEQ ID NO: 7 (e in FIG. 7B), the first phage having the genomic DNA sequence of SEQ ID NO: 1 (f in FIG. 7B), the third phage having the genomic DNA sequence of SEQ ID NO: 3 (g in FIG. 7B), and the phage having the genomic DNA sequence of SEQ ID NO: 8 (h in FIG. 7B).

### (Results)

An example of the results is shown in FIG. 7. When a phage combination exhibits bacteriolytic activity, the bacterial lawn formed on the plate turns clear only in an area to which the purified phage liquid has been dropped. It was found that all the combinations of two phages tested exhibited at least as much bacteriolytic activity as each phage used alone (FIG. 7).

From the above results, it was confirmed that the phage of one or more embodiments of the present invention is useful when used alone or in combination.

### Example 6: Bacteriolytic Activity of Combination of Obtained Bacteriophages (3)

### (Objective)

To verify the effect of a novel bacteriophage, which has been shown to have bacteriolytic activity against pathogenic bacteria of plant diseases when used alone, on plant diseases when used in combination.

### (Method)

### (1) Spot Test Method

A spot test was performed in accordance with the method described in “(4) Evaluation of host range of first phage” in Example 1. In this Example, strains of Xanthomonas cucurbitae (MAFF No. 673005), Xanthomonas campestris pv. vesicatoria (MAFF No. 301257) and Xanthomonas campestris pv. campestris (MAFF No. 106765), against which all the phages tested had been confirmed to exhibit bacteriolytic activity, were used.

As the purified phage liquids to be dropped, mixed solutions obtained by mixing equal amounts of the purified phage liquids prepared in Examples were used. Each mixed solution was prepared by appropriate dilution so that the total titer was equivalent to that in the case of single use. The phages used in this Example were as follows.

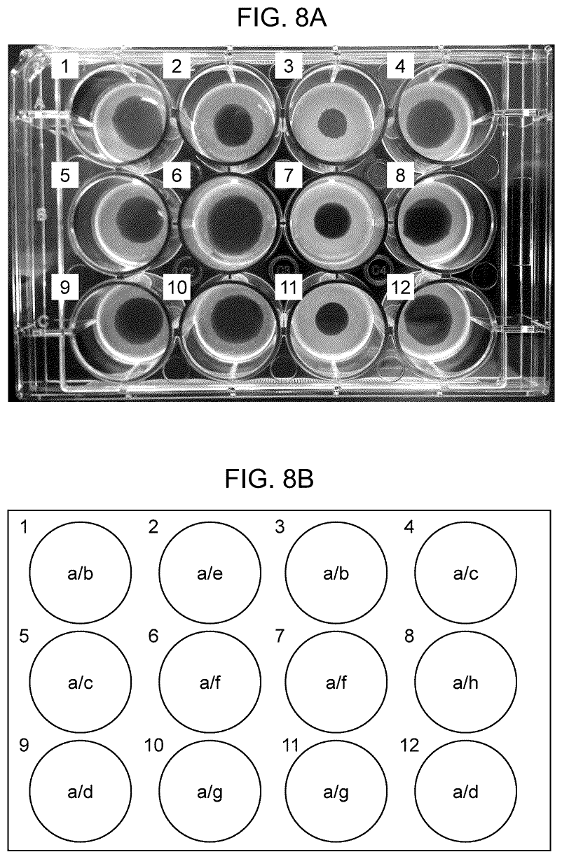

In this Example, the third phage having the genomic DNA sequence of SEQ ID NO: 3 (a in FIG. 8B) was used in combination with the phage having the genomic DNA sequence of SEQ ID NO: 4 (b in FIG. 8B), the phage having the genomic DNA sequence of SEQ ID NO: 5 (c in FIG. 8B), the phage having the genomic DNA sequence of SEQ ID NO: 6 (d in FIG. 8B), the phage having the genomic DNA sequence of SEQ ID NO: 7 (e in FIG. 8B), the first phage having the genomic DNA sequence of SEQ ID NO: 1 (f in FIG. 8B), the second phage having the genomic DNA sequence of SEQ ID NO: 2 (g in FIG. 8B), and the phage having the genomic DNA sequence of SEQ ID NO: 8 (h in FIG. 8B).

### (Results)

An example of the results is shown in FIG. 8. When a phage combination exhibits bacteriolytic activity, the bacterial lawn formed on the plate turns clear only in an area to which the purified phage liquid has been dropped. It was found that all the combinations of the two phages tested exhibited at least as much bacteriolytic activity as each phage used alone (FIG. 8).

From the above results, it was confirmed that the phage of one or more embodiments of the present invention is useful when used alone or in combination.

### Example 7: Test for Disease Control Effect on Tomato Bacterial Blight

### (Objective)

To verify the effect of an isolated novel bacteriophage having bacteriolytic activity against pathogenic bacteria of plant diseases on a plant disease when applied to a plant.

### (Method)

### (1) Preparation of Phage Spray Liquids

The phage spray liquid used in this test was prepared by the following procedure. First, a bacterial culture liquid of host bacteria for phage amplification was prepared by the following procedure. The host used was Xanthomonas campestris pv. vesicatoria (MAFF No. 301256), a strain against which all the phages tested had been confirmed to exhibit bacteriolytic activity. The bacterial cells were inoculated into YPG Broth and incubated overnight in a shaker set at 25° C. After the incubation, OD₆₀₀ (turbidity at a wavelength of 600 nm) was measured, and one having an OD₆₀₀ of about 1.0 was used as a bacterial culture liquid in the following.

A liquid mixture obtained by mixing equal amounts of the prepared bacterial culture liquid and the purified phage liquid (having a titer of about 108 PFU/mL) containing one kind of phage prepared in Example 1 was inoculated into a 100-fold amount of YPG culture liquid, and incubated in a shaker set at 25° C. for about 8 to 12 hours, and the obtained culture liquid was recovered as a phage crude solution. To the recovered crude liquid, a 1/10 volume of chloroform was added, and after vigorous stirring, the mixture was centrifuged at ×8,000 g at 20° C. for 5 minutes, and the supernatant was collected. The collected supernatant was passed through a 0.2 μm filter, and the filtrate was used as a purified phage liquid.

As a phage spray liquid containing one kind of phage, a liquid obtained by diluting the purified phage liquid with sterilized tap water to have a titer of about 109 PFU/mL was used.

### (2) Preparation of Bacterial Spray Liquids

The bacterial culture liquid prepared in (1) was used for the preparation of the bacterial spray liquid used for the treatment of plant infection in this test. A bacterial culture liquid diluted about 10,000 times with sterilized tap water was applied to a YPG Agar plate and incubated in an incubator set at 25° C. for about 1 to 3 days. The colonies on the YPG Agar plate were suspended in sterilized tap water, and collected to obtain a bacterial suspension. The bacterial suspension was diluted with sterilized tap water to have a final OD₆₀₀ of about 0.5 to prepare a bacterial spray liquid.

### (3) Plant Specimens

Commercially available tomato seeds were sowed and grown in a greenhouse, and seedlings having 50 or more leaves were used as specimens for evaluation.

### (4) Phage Application and Infection Treatment

In a phage application group, the phage spray liquid was foliar sprayed to each specimen twice before and after the bacterial infection treatment twice at an interval of 2 to 3 days, and after 2 to 3 days, the bacterial spray liquid was foliar sprayed to infect the specimen with the bacteria. The specimens infected with the bacteria were allowed to stand in a high-humidity plastic greenhouse for about 2 days after the infection treatment. Thereafter, the phage spray liquid was foliar sprayed twice at an interval of 2 to 3 days. The untreated group was treated in the same manner as described above, except that sterilized tap water was used instead of the purified phage liquid.

### (5) Determination of Incidence Rate

After 16 days from the infection treatment, the incidence rate was examined in the untreated group and each phage-sprayed group.

Leaves having a leaf surface on which a characteristic of tomato bacterial blight appeared were determined as diseased leaves, and the ratio of the number of diseased leaves to the total number of leaves was calculated as an incidence rate. The relative incidence rate of the phage-applied group was calculated as a relative value where the incidence rate of the untreated group was taken as 100%.

### (Results)

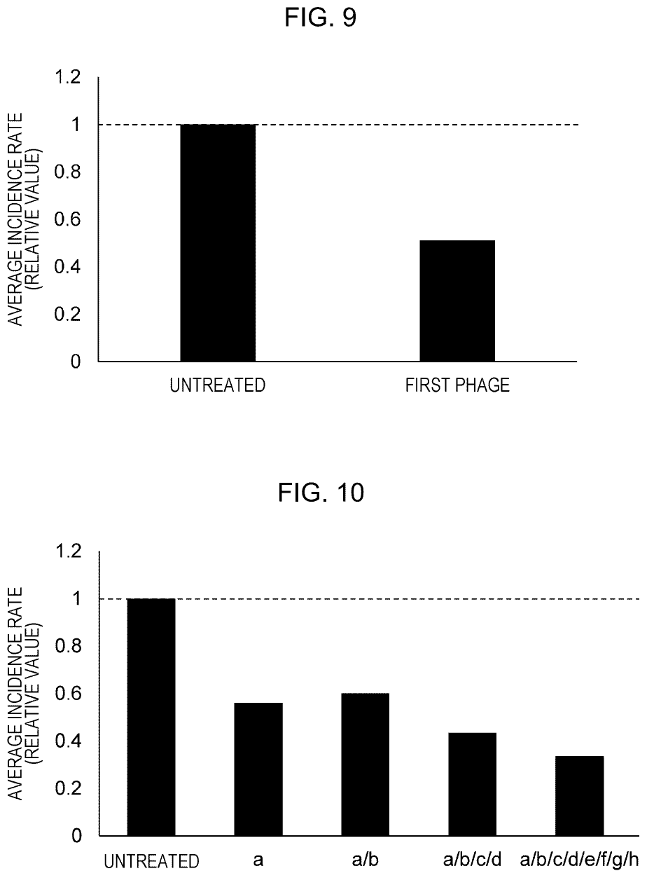

The results are shown in FIG. 9. The incidence rate in the untreated group was about 25%. On the other hand, the relative incidence rate was about 51.1% when the phage spray liquid containing the first phage was applied, with the incidence rate of the untreated group taken as 100%.

From the above, it was found that the phage of one or more embodiments of the present invention can effectively control a disease of a plant also when applied to the actual plant.

In the phage-applied group, there was no significant adverse effect on the plant considered to be caused by the phage, such as phytotoxicity on the leaves. From these results, it was found that the application of one or more embodiments of the phage of the present invention has few side effects and is an effective disease control means.

### Example 8: Test for Disease Control Effect on Broccoli Black Rot

### (Objective)

To verify the effect of an isolated novel bacteriophage having bacteriolytic activity against pathogenic bacteria of plant diseases on a plant disease when applied to a plant.

### (Method)

### (1) Preparation of Phage Spray Liquids

Phage spray liquids were prepared as described in Example 7, except that the bacteria used as the bacterial cells were Xanthomonas campestris pv. campestris (MAFF No. 106765), and the following phages were used.

As the phage spray liquids containing a plurality of types of phage, those obtained by mixing equal amounts of purified phage liquids adjusted to have titers of the same order and diluting the mixtures with sterilized tap water to have titers of about 109 PFU/mL were used.

In this Example, the third phage having the genomic DNA sequence of SEQ ID NO: 3 (a in FIG. 10) was used in combination with a phage having the genomic DNA sequence of SEQ ID NO: 9 (b in FIG. 10), or additionally with the phage having the genomic DNA sequence of SEQ ID NO: 8 (c in FIG. 10) and a phage having the genomic DNA sequence of SEQ ID NO: 10 (d in FIG. 10), or additionally with the phage having the genomic DNA sequence of SEQ ID NO: 5 (e in FIG. 10), the phage having the genomic DNA sequence of SEQ ID NO: 6 (f in FIG. 10), the phage having the genomic DNA sequence of SEQ ID NO: 7 (g in FIG. 10) and a phage having the genomic DNA sequence of SEQ ID NO: 11 (h in FIG. 10).

### (2) Preparation of Bacterial Spray Liquids

Phage spray liquids were prepared as described in Example 7, except that the bacterial cells were the bacteria described above.

### (3) Plant Specimens

Commercially available broccoli seeds were sowed and grown in a greenhouse, and seedlings having 5 or more leaves were used as specimens for evaluation.

### (4) Phage Application and Infection Treatment

Phage application and infection treatment were carried out as described in Example 7.

### (5) Determination of Incidence Rate

The determination was carried out as described in Example 7, except that the evaluation was carried out 16 days after the infection treatment.

### (Results)

The results are shown in FIG. 10. The incidence rate in the untreated group was about 52%. On the other hand, the relative incidence rate where the incidence rate of the untreated group was taken as 100% was about 56% when the phage spray liquid containing only the third phage alone was applied (“a” in FIG. 10).

Further, when phage spray liquids containing two or more kinds of phage were applied, the relative incidence rates were further reduced to about 45.7% on average. The relative incidence rate was about 60% when one type of phage was used in combination with the third phage (a total of two types) (“a/b” in FIG. 10), but in general, the disease control effect tended to be higher and the relative incidence rate tended to be lower as the number of types combined increased. To be specific, the relative incidence rate in the case of combining a total of four kinds was about 43.5% (“a/b/c/d” in FIG. 10), and the relative incidence rate in the case of combining a total of eight kinds was about 33.5% (“a/b/c/d/e/f/g/h” in FIG. 10).

From the above, it was found that the phage of one or more embodiments of the present invention can effectively control a disease of a plant regardless of the kind of the plant itself even when applied to an actual plant. In addition, it was found that a higher effect can be obtained by applying the phage of one or more embodiments of the present invention in combination.

In the phage-applied group, there was no significant adverse effect on the plant considered to be caused by the phage, such as phytotoxicity on the leaves. From these results, it was found that the application of one or more embodiments of the phage of the present invention has few side effects and is an effective disease control means.

All publications, patents and patent applications cited herein are incorporated herein by reference in their entirety.

### SEQUENCE LISTING

B230189 Sequence.xml

1. A method for controlling a plant disease, comprising contacting a bacteriophage to a target plant.

2. The method according to claim 1, wherein the bacteriophage has a genomic DNA sequence comprising a base sequence shown in SEQ ID NO: 1.

3. The method according to claim 1, wherein the bacteriophage has a genomic DNA sequence comprising a base sequence shown in SEQ ID NO: 2.

4. The method according to claim 1, wherein the bacteriophage has a genomic DNA sequence comprising a base sequence shown in SEQ ID NO: 3.

5. The method according to claim 2, wherein the bacteriophage is bacteriolytic against a bacterium of genus Xanthomonas.

6. The method according to claim 3, wherein the bacteriophage is bacteriolytic against a bacterium of genus Xanthomonas.

7. The method according to claim 4, wherein the bacteriophage is bacteriolytic against a bacterium of genus Xanthomonas.

8. The method according to claim 1, wherein the bacteriophage is contained in a plant disease control composition.

9. The method according to claim 8, wherein the plant disease control composition comprises another bacteriophage exhibiting bacteriolytic activity against a bacterium of genus Xanthomonas.

10. A method for identifying a bacterium of genus Xanthomonas, comprising:
culturing a test bacterium isolated from a plant tissue affected by a plant disease to obtain a culture;
mixing the culture with a bacteriophage to obtain a mixture;
culturing the mixture under a predetermined condition; and
determining that the test bacterium is a bacterium of genus Xanthomonas when the test bacterium is lysed after the mixture is cultured under the predetermined condition.

11. The method according to claim 10, wherein the mixture is cultured on a solid culture medium and wherein the mixture further comprises a soft agar-containing liquid culture medium.

12. The method according to claim 10, wherein in the culture is cultured on a solid culture medium, and wherein the culture comprises a soft agar-containing liquid culture medium.

13. The method according to claim 10, wherein the test bacterium is isolated from the plant tissue affected by the plant disease before culturing the test bacterium.

14. The method according to claim 10, wherein the bacteriophage has a genomic DNA sequence comprising a base sequence shown in SEQ ID NO: 1.

15. The method according to claim 10, wherein the bacteriophage has a genomic DNA sequence comprising a base sequence shown in SEQ ID NO: 2.

16. The method according to claim 10, wherein the bacteriophage has a genomic DNA sequence comprising a base sequence shown in SEQ ID NO: 3.

17. The method according to claim 14, wherein the bacteriophage is bacteriolytic against a bacterium of genus Xanthomonas.

18. The method according to claim 15, wherein the bacteriophage is bacteriolytic against a bacterium of genus Xanthomonas.

19. The method according to claim 16, wherein the bacteriophage is bacteriolytic against a bacterium of genus Xanthomonas.

20. The method according to claim 10, wherein the bacteriophage is contained in a bacteriolytic agent.
